(19) United States
(12) Patent Application Publication
(10) Pub. No.: US 2025/0084420 A1
(43) Pub. Date: Mar. 13, 2025

(54) COMPOSITIONS AND METHODS FOR TREATING CMT1A OR CMT1E DISEASES WITH RNAI MOLECULES TARGETING PMP22

(71) Applicants: UNIVERSITE DE MONTPELLIER, Montpellier (FR); INSTITUT NATIONAL DE LA SANTE ET DE LA RECHERCHE MEDICALE (INSERM), Paris (FR)

(72) Inventor: Nicolas TRICAUD, Montpellier (FR)

(73) Assignees: UNIVERSITE DE MONTPELLIER, Montpellier (FR); INSTITUT NATIONAL DE LA SANTE ET DE LA RECHERCHE MEDICALE (INSERM), Paris (FR)

(21) Appl. No.: 18/294,325

(22) PCT Filed: Aug. 2, 2022
(86) PCT No.: PCT/EP2022/071712

§ 371 Date: Feb. 1, 2024

(30) Foreign Application Priority Data
Aug. 2, 2021 (EP) 21306077.5

(51) Int. Cl. C12N 15/113; A61P 25/02; C12N 15/86

(52) U.S. Cl. CPC C12N 15/1138; A61P 25/02; C12N 15/86; C12N 2310/11; C12N 2310/531; C12N 2750/14143

(57) ABSTRACT

RNA interferent (RNAi) molecules that inhibit a PMP22 protein expression and/or activity by targeting exon 5 of a nucleic acid sequence encoding the PMP22 protein, and the prevention and treatment of the Charcot-Marie-Tooth type 1A or 1E diseases in which an adeno-associated virus (AAV) vector including an RNAi molecule is administered.

### FIELD

The present invention relates to the fields of biopharmaceuticals and therapeutics composed of nucleic acid-based molecules. Further, the present invention relates to RNA interferent molecules targeting PMP22 and their use for the treatment of Charcot-Marie-Tooth 1A (CMT-1A) or 1E (CMT-1E) diseases.

### BACKGROUND

Charcot-Marie-Tooth (CMT) disease is a demyelinating disorder of the peripheral nervous system, characterized by progressive weakness and atrophy, initially of the leg muscles and later of the distal muscles of the arms. Charcot-Marie-Tooth disease is classified in two main groups on the basis of electrophysiologic properties and histopathology: primary peripheral demyelinating neuropathies (designated CMT1 when they are dominantly inherited) and primary peripheral axonal neuropathies (CMT2).

Demyelinating neuropathies are characterized by severely reduced nerve conduction velocities (less than 38 m/sec), segmental demyelination and remyelination with onion bulb formations on nerve biopsy, slowly progressive distal muscle atrophy and weakness, absent deep tendon reflexes, and hollow feet.

Alterations in Peripheral Myelin Protein 22 (PMP22) expression are associated with demyelinating peripheral neuropathies.

The protein encoded by the PMP22 gene is a glycoprotein of 160 amino acids and constitutes 2-5% of overall peripheral myelin proteins (NCBI Reference Sequence: NP_000295.1). The protein PMP22 is an integral membrane glycoprotein of the internodal myelin (https://en.wikipedia.org/wiki/Peripheral_myelin_protein_22). Predicted structure of the PMP22 protein comprises four transmembrane domains, two extracellular loops, and cytoplasmic N- and C-terminal tails. PMP22 protein is essential for the compactness and stability of peripheral myelin and is also involved in the proliferation and apoptosis of Schwann cells (Liao et al., Sci Rep 7, 15363 (2017)). In human genome, PMP22 is located within the chromosome 17p11.2. PMP22 is a 40 kb gene that consists of six exons conserved in both humans and rodents. The coding region of the PMP22 spans from exon-2 to exon-5. Exon-2 encodes the first transmembrane domain of PMP22. Exon-3 encodes the first extracellular loop. Exon-4 encodes the second transmembrane domain and half of the third transmembrane domain. Exon-5 encodes the remaining half of the third transmembrane domain, the second extracellular domain, the fourth transmembrane domain, and the 3′ untranslated region (Li et al., Mol Neurobiol. 2013; 47(2):673-698).

PMP22 related diseases disrupt the organization of myelin, and subsequently axonal integrity, which is responsible for the disabilities in patients with PMP22 mutations.

CMT carries a prevalence of one in 2,500 people and mutations of PMP22 are responsible for >50% cases of CMT (Li et al., Mol Neurobiol. 2013; 47(2):673-698). Duplication of a 1.5-Mb DNA segment on chromosome 17p11.2-12 encompassing the PMP22 gene in Schwann cells results in a deficit of myelination in peripheral nerves leading to Charcot-Marie-Tooth disease type 1 A (CMT-1A), which is an autosomal dominant demyelinating neuropathy and the most common subtype of CMT. The same 1.5-Mb DNA segment triplication causes a more severe demyelinating polyneuropathy, whereas large deletion at the same segment results in hereditary neuropathy with liability to pressure palsies (HNPP) (Liao et al., Sci Rep 7, 15363 (2017)). Point mutations causing CMT-1E are relatively rare, causing around 1% of cases of CMT (Jerath et al., Biochim Biophys Acta. 2015; 1852(4):667-678). Two mutations at glycine 94, a single guanine insertion or deletion in PMP22, result in different reading frameshifts and, consequently, an extended G94fsX222 or a truncated G94fsX110 protein, respectively. Both of these autosomal dominant mutations alter the second half of PMP22 and yet are linked to clinical phenotypes with distinct severities. The G94fsX222 and G94fsX110 causes severe neuropathy diagnosed as Dejerine-Sottas disease or Charcot-Marie-Tooth disease type 1E (Johnson et al., J Neurosci Res. 2005 Dec. 15; 82(6):743-52).

So far, there is no cure for Charcot-Marie-Tooth disease, and supportive treatments are limited to physical therapy, orthotics, surgical treatment of skeletal and soft tissue abnormalities, as well as symptomatic drug treatment. Nonetheless, numerous potential therapies are under investigation.

Most existing approaches are based on small molecules. An approach based on gene therapy using AAV is currently investigated by SAREPTA Therapeutics. However, this approach aims at increasing the production of Neurotrophin 3 and targets the muscles and consequences of the disease and is not directed towards the myelin of the nerves.

Some approaches tried to restore a normal level of PMP22 protein. However, such strategy faces multiple challenges, in particular achieving a level of PMP22 that is not too high or not too low since excessive or deficient level of this protein can result in peripheral neuropathy.

Zhao et al., (J Clin Invest. 2018) describes antisense oligonucleotides (DNA) targeting the 3′ UTR of the human PMP22 mRNA and decreasing the expression of the human PMP22 protein and their effects in CMT-1A rat and mouse models.

Serfecz et al. (Gene Ther. 2019) describes an endogenous microRNA 29a targeting the 3′ UTR of the human PMP22 mRNA, a region about 300 bp upstream from the PolyA signal, and decreasing the expression of the human PMP22 protein in HEK 293 cells transfected to express hPMP22.

Boutary et al. (Commun Biol. 2021) and WO 2020/064749 describe a small inhibitory RNA (siRNA) coupled to squalene and targeting the 3′ UTR of human and mouse PMP22 mRNA and decreasing mouse and human PMP22 expression in mouse model of CMT-1A and resulting in functional effects in these animals.

Gautier et al. (Nature Comm., 2021) describes 2 shRNAs targeting human PMP22 mRNA in the coding sequence exons 3 and 4 and decreasing the expression of human PMP22 in HEK 293 cells transfected with human PM22.

There is a need to provide further solutions for the prevention and/or treatment of CMT-1A or 1E diseases.

There is a need to have a drug candidate for the prevention and/or treatment of CMT-1A or 1E diseases which can target the various forms of the PMP22 protein.

There is a need to have a drug candidate able to restore a normal level of PMP22 protein.

There is a need to have a drug candidate for the prevention and/or treatment of CMT-1A or CMT-1E diseases which can target the full length and the truncated forms of the PMP22 protein.

There is a need to have a drug candidate for the prevention and/or treatment of CMT-1A or CMT-1E diseases which can efficiently reduce or inhibit the expression of the PMP22 mRNA or protein.

There is a need to have a drug candidate for the prevention and/or treatment of CMT-1A or CMT-1E diseases which can be administered by general or local route.

There is a need to have a drug candidate for the prevention and/or treatment of CMT-1A or CMT-1E diseases which can be administered by systemic route or by local route, for example by intranerve route.

There is also a need to have drug candidate for the prevention and/or treatment of CMT-1A or CMT-1E diseases that can be easily produced at industrial scale level.

There is also a need to have drug candidate for the prevention and/or treatment of CMT-1A or CMT-1E diseases which can be easily and cost-effectively developed in preclinical model without the need to be adapted or modified for moving into human clinical development.

The present invention has for purpose to satisfy all or part of those needs.

As shown in the Examples, the inventors have surprisingly observed that by targeting exon 5, for example the coding sequence in exon 5, of a nucleic acid sequence encoding said PMP22 protein it was possible to develop RNAi molecules able to efficiently prevent the expression of the full length as well as the truncated forms of the protein. Indeed, other RNAi molecules targeting other exons of the PMP22 protein were not able to prevent the expression of the truncated forms.

Further, as shown in the Examples, the RNAi molecules of the invention were proven to be advantageously efficient on preventing the expression of the cynomolgus (Macaca fascicularis) PMP22 protein. Therefore, the RNAi molecules of the invention can be advantageously used and developed on a cynomolgus model at the preclinical stages and can thereafter be translated into human development without the need to be adapted or modified. This allows a substantial advantage in terms of costs and speed of development.

Also, as indicated in the Examples, the RNAi of the invention can be produced with high yield allowing an efficient industrial scale-up.

Further, the RNAi molecules of the invention, can be advantageously implemented in a delivery system. A delivery system may be an expression vector, for example a non-viral or a viral vector, such as an adeno-associated virus, allowing the transduction of the RNAi molecules in nerve cells, such as Schwann cells.

It was further observed that the RNAi disclosed herein, when packaged in an adeno-associated virus, can be produced with high yield allowing an efficient industrial scale-up.

Advantageously, the RNAi molecules of the invention allows developing a gene therapy based on an AAV9 vector administered directly into the nerve, to treat an inherited disease of the nerve: CMT1A or CMT1E. The therapeutic vector expresses a small inhibitory RNA (shRNA) specifically decreasing the expression of the PMP22 protein and preventing the disease.

Advantageously, the RNAi molecules of the invention were shown to be efficient to inhibit the expression of the human protein and of the cynomolgus (Macaca fascicularis) protein and to be efficiently produced in expression viral vectors. Those features make the RNAi molecules of the invention suitable for the development of a gene therapy product for CMT1A or CMT1E.

Starting from a pool of various RNAi molecules, the inventors have shown that targeting exon 5 of the PMP22 protein was a particularly interesting strategy for reducing or suppressing the expression of PMP22 in its full-length as well as N-terminally truncated forms for the treatment and prevention of the CMT1A or 1E diseases (see Example 2).

Then, they designed and tested 33 RNAi molecules targeting the region of exon 5 of the PMP22 protein. Among this pool, they surprisingly identified 6 effective RNAi molecules (RNAi #11, #12, #16, #17, #18 and #22) that efficiently suppressed or reduced the expression of hPMP22 in its full-length as well as N-terminally truncated forms (see Example 3).

Among those 6 effective RNAi molecules, the inventors then showed that 2 RNAi molecules (RNAi #16 and #17) were surprisingly and advantageously efficient to reduce or suppress the expression of cynomolgus PMP22 (see Examples 5 and 6), which constitutes an industrial advantage.

Finally, during production of AAV9 viral particles comprising either RNAi #16 or RNAi #17, the inventors surprisingly observed that adeno-associated vector yield production was increased with RNAi #17 compared to RNAi #16, giving to this compound a further advantage in terms of industrial production (see Example 7).

Then, the inventors confirmed, in an in vivo mouse model, that intranerve injection of AAV9 viral particles expressing RNAi #17 presented significant protective efficacy on CMT1A neuropathy targeting PMP22 overexpression in Schwann cells by increasing neuromuscular and electrophysiological performances in C3 mouse model of CMT1A (see Example 8).

Finally, they showed that AAV9 viral particles expressing RNAi #17 were able to reduce PMP22 expression level in sciatic nerves of cynomolgus monkeys, following bilaterally delivery in the sciatic nerves of 3 adult cynomolgus monkeys through intraneural perifascicular (INPF) injections (see Example 9).

Thus, it was surprisingly found by the Applicants that, among the 33 tested RNAi molecules targeting exon 5 of the PMP22 protein, the particular RNAi #17 (having the antisense sequence of SEQ ID NO: 61) was the only RNAi molecule that:
efficiently suppressed or reduced the expression of hPMP22 in its full-length as well as N-terminally truncated forms,
efficiently reduced or suppressed the expression of cynomolgus PMP22,
presented a high adeno-associated vector yield production,
presented significant protective efficacy on CMT1A neuropathy targeting PMP22 overexpression in Schwann cells by increasing neuromuscular and electrophysiological performances in a mouse model of CMT1A, and
was able to reduce PMP22 expression level in sciatic nerves of cynomolgus monkeys, following bilaterally delivery in the sciatic nerves of adult cynomolgus monkeys.

### SUMMARY

A first aspect of the invention relates to an adeno-associated virus (AAV) vector comprising an RNAi molecule comprising an antisense oligonucleotide of sequence SEQ ID NO: 61, and/or an oligonucleotide coding for an antisense oligonucleotide of sequence SEQ ID NO: 61.

According to some embodiments, the RNAi molecule is a shRNA or a microRNA.

According to some embodiments, the RNAi molecule inhibits a PMP22 protein expression and/or activity.

According to some embodiments, the AAV vector is selected from the group consisting of AAV9, AAV2/9, AAV10, AAVrh10 and AAV2/rh10.

According to some embodiments, the AAV vector is an AAV serotype 9 (AAV9).

According to some embodiments, the AAV vector is a single-stranded AAV or a self-complementary AAV.

A second aspect of the invention relates to an isolated host cell containing an AAV vector as described herein.

A third aspect of the invention relates to a pharmaceutical composition comprising an AAV vector as described herein, and a pharmaceutically acceptable excipient.

Another aspect of the invention relates to an AAV vector, or a pharmaceutical composition, as described herein for use as a medicament.

Another aspect of the invention relates to an AAV vector, or a pharmaceutical composition, as described herein for use in preventing and/or treating a Charcot-Marie-Tooth type 1A or a Charcot-Marie-Tooth type 1E disease in a patient in need thereof.

According to some embodiments, the AAV vector or the pharmaceutical composition is to be administered by systemic, intrathecal or intraneural route.

### BRIEF DESCRIPTION OF THE DRAWINGS

FIG. 1 shows positions of the RNAi molecules—grey shaded letters: shRNA1 (sh1 targeting CCTGTTCTTCTGCCAACTCIT (SEQ ID NO: 34) in Exon 4), shRNA2 (sh2 targeting GGCAATGGACACGCAACTGAT (SEQ ID NO: 35) in Exon 3), shRNA3 (sh3 targeting CGGTGTCATCTATGTGATCIT (SEQ ID NO: 36) in Exon 5), shRNA4 (sh4 targeting TGTCGATCATCTTCAGCATTC (SEQ ID NO: 37) in Exon 4) and shRNA5 (sh5 targeting CACGATCGTCAGCCAATGGAT (SEQ ID NO: 38) in Exon 2-3) against the mRNA hPMP22 sequence (SEQ ID NO: 79—NCBI Reference Sequence: NM_000304.4). Sequence of exon 5 targeted by the RNAi disclosed herein is underlined (CCCTGGCCCTTCTCAGCGGTGTCATCTATGTGATCTTGCGGAAACGCGAAT GA (SEQ ID NO: 80), from nucleotide 638 to 690 in the NM_000304.4 sequence). Low case: non-coding sequence. High case: coding sequence. Exon 1: courier italic regular. Exon 2: courier italic bold. Exon 3: courier regular. Exon 4: courier regular bold. Exon 5: arial regular.

FIG. 2 shows a representative Western Blot showing the level of expression of hPMP22-Flag in HEK293 cells co-transfected with a human-flag vector pCMV3-hPMP22-Flag and the different RNAi molecules comprising sense and antisense nucleic acid sequences: shRNA1-5 (see TABLE OF SEQUENCES for sense and antisense nucleic acid sequences). β-actin was used as internal reference. The Western Blot shows that shRNA3 was able to prevent the expression of full-length and truncated hPMP22.

FIG. 3 shows normalized band intensities of hPMP22-Flag in HEK293 cells co-transfected with a human-flag vector pCMV3-hPMP22-Flag and the different RNAi molecules comprising sense and antisense nucleic acid sequences: RNAi #1 to #33 (see TABLE OF SEQUENCES for sense and antisense nucleic acid sequences) measured on a Western Blot. Expression of the level of the proteins were normalized over the level of hPMP22-Flag measured in cells co-transfected with shRNA scramble (control). shRNA3 was used as positive control. An at least 2.5-fold reduction in hPMP22-Flag expression was set as a threshold.

FIG. 4 shows an alignment of sequence between the targeted sequence in exon 5 of human-PMP22 (hPMP22) and the corresponding sequence in cynomolgus-PMP22 (cPMP22—Macaca fascicularis).

FIG. 5 shows normalized band intensities of cPMP22-Flag in HEK293 cells co-transfected with a cynomolgus-flag vector pCMV3-cPMP22-Flag and the different RNAi molecules: RNAi #11, #12, #16, #17, #18 and #20 (see TABLE OF SEQUENCES for sense and antisense nucleic acid sequences) measured on a Western Blot. Expression of the level of the proteins were normalized over the level of cPMP22-Flag measured in cells co-transfected with shRNA scramble (control). shRNA3 was used as positive control.

FIG. 6A: represents a normalized cynomolgus flag-PMP22 band intensity for shRNA control and RNAi #16 measured on a Western Blot.

FIG. 6B: represents normalized human flag-PMP22 band intensity for shRNA control and RNAi #16 measured on a Western Blot. Expression of the level of the proteins were normalized over the level of PMP22-Flag measured in cells co-transfected with shRNA scramble (control).

FIG. 7A: represents a normalized cynomolgus flag-PMP22 band intensity for shRNA control and RNAi #17 measured on a Western Blot.

FIG. 7B: represents normalized human flag-PMP22 band intensity for shRNA control and RNAi #17 measured on a Western Blot. Expression of the level of the proteins were normalized over the level of PMP22-Flag measured in cells co-transfected with shRNA scramble (control).

FIG. 8 is a graph showing the production yield of AAV9 viral particles expressing RNAi #16 (AAV9 shRNA 16), RNAi #17 (AAV9 shRNA 17) or control AAV9 viral particles by HEK293 cells. Amounts are represented as viral particles per mL.

FIG. 9 is a graph showing the decrease of human PMP22 expression induced by AAV9 expressing RNAi #17 (AAV9-sh17). Control AAV9 or AAV9-sh17 at three different doses (4.2×10⁹ vg/nerve, 1.7×10¹⁰ vg/nerve and 3.4×10¹⁰ vg/nerve) were administrated through a single bilateral intraneural injection in C3 CMT1A humanized mice sciatic nerves 4 days after birth. Western blot analysis of PMP22 expression in sciatic nerves was performed 2 months after injection. “*” signs represent statistical differences compared to non-injected control; “#” signs represent statistical differences compared to control AAV9 viral particles (control AAV9).

FIGS. 10A-10D are a collection of graphs showing the effect of AAV9 expressing RNAi #17 (AAV9-sh17) injection on rotarod latency, grip strength, nerve conduction velocity and CMAP amplitude in C3 CMT1A humanized mice. Control AAV9 or AAV9-sh17 at three different doses (4.2×10⁹ vg/nerve, 1.7×10¹⁰ vg/nerve and 3.4×10¹⁰ vg/nerve) were administrated through a single bilateral intraneural injection in C3 CMT1A humanized mice sciatic nerves 4 days after birth. Rotarod latency (10A), grip strength (10B), nerve conduction velocity (10C) and CMAP amplitude (10D) were measured on 2-month-old mice. “*” signs represent statistical differences compared to non-injected control; “#” signs represent statistical differences compared to control AAV9 viral particles (control AAV9).

FIG. 11 is a graph showing PMP22 expression in sciatic nerves of cynomolgus monkeys injected either with control AAV9 vector, or different doses of AAV9 vector expressing RNAi #17 (AAV9-sh17) (2×10¹² vg/nerve or 5×10¹² vg/nerve) as indicated. PMP22 concentration was measured by Western blot 28 or 29 days after injection and normalized over β-tubulin concentration, considered as control protein (constant housekeeping protein).

### DETAILED DESCRIPTION

In the present invention, the following terms have the following meanings:

Unless otherwise required by context, singular terms shall include pluralities and plural terms shall include the singular.

It must be noted that as used herein and in the appended claims, the singular forms “a”, “an”, and “the” include plural referents unless the context clearly dictates otherwise. Thus, for example, reference to “an RNAi molecule” includes a plurality of such RNAi molecules, and so forth.

The terms “about” or “approximately” as used herein refer to the usual error range for the respective value readily known to the skilled person in this technical field. Reference to “about” a value or parameter herein includes (and describes) embodiments that are directed to that value or parameter per se. In some embodiments, the term “about” refers to ±10% of a given value. However, whenever the value in question refers to an indivisible object, such as a molecule or other object that would lose its identity once subdivided, then “about” refers to ±1 of the indivisible object.

It is understood that aspects and embodiments of the present disclosure described herein include “having,” “comprising,” “consisting of,” and “consisting essentially of” aspects and embodiments. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the basic and novel characteristic(s) of the disclosure. It is understood that the different embodiments of the disclosure using the term “comprising” or equivalent cover the embodiments where this term is replaced with “consisting of” or “consisting essentially of”.

As used herein the term “AAV9 vector” has its general meanings in the art and refers to a vector derived from an adeno-associated virus serotype 9. In particular, the term “AAV9”, as used herein, refers to a serotype of adeno-associated virus with a genome sequence as defined in the GenBank accession number AAS99264.

The terms “complementary”, “fully complementary” and “substantially complementary” herein may be used with respect to the base matching between the sense strand and the antisense strand of an RNAi molecule, such as a dsRNA, or between the antisense strand of an RNAi molecule and a target sequence, as will be understood from the context of their use.

The terms “delivery system” as used herein intends to refer a system comprising a nucleic acid-based expression system, such as a plasmid, that controls the expression of a nucleic acid sequence of interest, such as an RNAi molecule, within a targeting cell, the nucleic acid sequence of interest, and a nucleic acid sequence delivery system, such as a virus or a liposome, that controls the delivery of the nucleic acid-based expression system in a cell.

By “inhibit”, “down-regulate”, “silence”, “reduce”, or “suppress”, it is meant that an expression of the gene, or a level of RNA molecules or equivalent RNA molecules encoding a PMP22 protein, or a level of activity of a PMP22 protein, is at least partially reduced or suppressed to below that observed in the absence of an RNAi molecule (e.g., siRNA) of the invention. The degree of inhibition can be greater than 40%, 50%, 60%, 70%, 80%, 90%, 95%, or 99%.

As used herein, the term “isolated” used in conjunction with a given item, such as a nucleic acid sequence, an RNAi molecule, or an expression vector, intends to mean that this item is not associated with all or a portion of the matter with which it is associated with in nature.

The terms “level of expression” or “expression level” are used generally to refer to the amount of a polynucleotide, a polypeptide, an amino acid product or protein in a biological sample.

The terms “nucleic acid sequence”, “oligonucleotide”, or “polynucleotide” are used interchangeably and intend to mean a polymeric form of naturally occurring or modified nucleic acids or nucleotides that are at least 10 bases in length. In certain embodiments, the bases may be ribonucleotides or deoxyribonucleotides or a modified form of either type of nucleotide. The term includes single and double stranded forms of DNA or RNA.

As used herein, the term “nucleotide” is defined as a modified or naturally occurring deoxyribonucleotide or ribonucleotide. Nucleotides typically include purines and pyrimidines, which include thymidine (T), cytidine (C), guanosine (G), adenosine (A) and uridine (U).

The term “modified nucleotide” includes nucleotide with modified or substituted sugar groups, or morpholino moieties rather than ribose or deoxyribose moieties, and the like.

Within the meaning of the invention, the terms “patient”, “subject”, “individual” or “recipient” are used interchangeably and intend to refer preferably to a mammal in need of a therapeutic or prophylactic treatment. Mammals include, but are not limited to, domesticated animals (e.g., cows, sheep, cats, dogs, and horses), primates (e.g., humans and non-human primates such as monkeys), rabbits, and rodents (e.g., mice and rats). In some exemplary embodiments, the individual or recipient is a human.

As used herein, the terms “prevent”, “preventing” or “delay progression of” (and grammatical variants thereof) with respect to a disease or disorder relate to prophylactic treatment of the disease or the disorder, e.g., in an individual suspected to have the disease or the disorder, or at risk of developing the disease or the disorder. Prevention may include, but is not limited to, preventing or delaying onset or progression of the disease and/or maintaining one or more symptoms of the disease or disorder at a desired or sub-pathological level. The term “prevent” does not require the 100% elimination of the possibility or likelihood of occurrence of the event. Rather, it denotes that the likelihood of the occurrence of the event has been reduced in the presence of a composition or method as described herein.

The term “RNA interferent molecule” as used herein intends to refer to a single or double-stranded ribonucleic acid, possibly comprising one or more modified ribonucleotides, complementary to at least a portion of a messenger RNA (mRNA) and whose interference with which results in the degradation of this mRNA and the reduction or suppression of the corresponding protein expression. RNA interferent molecules include miRNA, dsRNA, siRNA or shRNA.

Within the disclosure, the term “significantly” used with respect to change intends to mean that the observe change is noticeable and/or it has a statistic meaning.

Within the disclosure, the term “substantially” used in conjunction with a feature of the disclosure intends to define a set of embodiments related to this feature which are largely but not wholly similar to this feature. The difference between the set of embodiments related to a given feature and the given feature is such that in the set of embodiments, the nature and function of the given feature is not materially affected.

As used herein, the terms “target” or “targeting” may refer to a nucleic acid sequence able to specifically bind to a PMP22 gene or a PMP22 mRNA encoding a PMP22 gene product. In particular, it may refer to a nucleic acid sequence able to inhibit said gene or said mRNA by methods known to the skilled in the art (e.g., antisense, RNA interference). As used herein, “target sequence” may refer to a contiguous portion of the nucleotide sequence of the PMP22 gene or of an mRNA molecule formed during the transcription of the PMP22 gene.

As used herein, the terms “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment.

A “therapeutically effective amount” is intended for a minimal amount of active ingredient (e.g., RNAi molecule or expression vector comprising an RNAi molecule according to the invention) which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific compound employed; and like factors well known in the medical arts.

By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria, e.g., disease manifestation, etc.)

The terms “vector” and “nucleic acid sequence delivery system” are used interchangeably. They are used herein to refer to transport exogenous nucleic acid molecules to a target cell or tissue. They are used to refer to any vehicle (e.g., nucleic acids, plasmid, or virus) used and capable of facilitating the transfer of an RNAi molecule as disclosed herein to a host cell or a target cell.

The term “expression vector” refers to a vector that is suitable for transformation of a host cell or a target cell and contains nucleic acid sequences comprising control sequences that direct and/or control the expression of inserted nucleic acid sequences. The term “expression” includes, but is not limited to, processes such as transcription.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention. All publications mentioned herein are incorporated herein by reference to disclose and describe the methods and/or materials in connection with which the publications are cited.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” the list of items “and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the description herein.

In some embodiments, interferent RNA (RNAi) molecules of the invention are able to target exon 5 of the nucleic acid sequence encoding a PMP22 mRNA.

In some embodiments, the interferent RNA (RNAi) molecules of the invention are able to inhibit the expression and/or activity of the PMP22 protein.

In some embodiments, an RNAi molecule according to the invention comprises at least one antisense nucleic acid sequence.

In some embodiments, the RNAi molecule according to the invention comprises, or consists of, an antisense oligonucleotide of sequence SEQ ID NO: 61, and/or an oligonucleotide coding for an antisense oligonucleotide of sequence SEQ ID NO: 61.

In some embodiments, the RNAi molecule according to the invention comprises an antisense oligonucleotide having the sequence of SEQ ID NO: 61, and/or an oligonucleotide coding for an antisense oligonucleotide having the sequence of SEQ ID NO: 61.

In some embodiments, the RNAi molecule according to the invention is an antisense oligonucleotide having the sequence of SEQ ID NO: 61, and/or an oligonucleotide coding for an antisense oligonucleotide having the sequence of SEQ ID NO: 61.

In some embodiments, the RNAi molecule according to the invention comprises at least one antisense oligonucleotide, wherein the sequence of said at least one antisense oligonucleotide consists of the sequence SEQ ID NO: 61.

In some embodiments, the RNAi molecule according to the invention comprises at least one antisense nucleic acid sequence, wherein said antisense nucleic acid sequence consists of the sequence SEQ ID NO: 61.

In some embodiments, the RNAi molecule according to the invention comprises at least one antisense oligonucleotide coding sequence, wherein said at least one antisense oligonucleotide coding sequence consists of the sequence SEQ ID NO: 61.

In some embodiments, the RNAi molecule according to the invention comprises at least one antisense nucleic acid coding sequence, wherein said antisense nucleic acid coding sequence consists of the sequence SEQ ID NO: 61.

In some embodiments, the RNAi molecule according to the invention comprises a sequence encoding an antisense oligonucleotide, wherein said sequence encoding an antisense oligonucleotide consists of the sequence SEQ ID NO: 61.

In some embodiments, the RNAi molecule according to the invention comprises a sequence encoding an antisense nucleic acid sequence, wherein said sequence encoding an antisense nucleic acid sequence consists of the sequence SEQ ID NO: 61.

In some embodiments, a nucleic acid sequence encoding a PMP22 protein is a gene sequence or an mRNA.

PMP22 refers to Peripheral Myelin Protein 22 which is involved in growth regulation, and in myelinization in the peripheral nervous system. The human PMP22 sequence is available from UniProtKB database under reference Q01453 (PMP22_HUMAN). The mRNA transcript of the human PMP22 protein (SEQ ID NO: 79) is available from NCBI database under the NCBI Reference Sequence: NM_000304.4.

In some embodiments, the RNAi molecules of the invention are able to inhibit the expression and/or activity of the full length and/or of N-terminally truncated forms the PMP22.

In some embodiments, the RNAi molecules of the invention are able to inhibit the expression and/or activity of the human or the cynomolgus PMP22 protein. In some embodiments, the RNAi molecules of the invention inhibit the expression and/or activity of the human PMP22 protein.

The PMP22 protein may be expressed in a nerve cell. A nerve cell may be selected in the group consisting of a Schwann cell and a neuron.

An RNAi molecule of the invention may target a region comprising or consisting in the nucleic acid sequence ranging from position 638 to position 690 of SEQ ID NO: 79 (mRNA transcript: NCBI Reference Sequence: NM_000304.4).

An antisense nucleic acid sequence may comprise or consist of from about 10 to about 50 nucleotides, from about 12 to about 35 nucleotides, from about 12 to about 30, from about 12 to about 25, from about 12 to about 22, from about 15 to about 35, from about 15 to about 30, from about 15 to about 25, from about 15 to about 22, or from about 18 to about 22, for example of about 19, about 20 or about 21 nucleotides.

An antisense nucleic acid sequence may comprise one or two single-stranded overhangs. A single-stranded overhang may be a 3′ overhang or a 5′ overhang. The 3′ and/or 5′ overhang may consist of at least one, preferably at least two deoxyribonucleotides T (referred to as “dT”). For example, the 3′ and/or 5′ overhang(s) may consist of two deoxyribonucleotides T.

An antisense nucleic acid sequence may comprise one or two 3′ overhangs.

An antisense nucleic acid sequence may comprise or consist of 19, 20 or 21 base pairs.

It is understood to the skilled in the art that inhibiting expression of a gene or a protein, typically results in a decrease or even abolition of the gene product, the polypeptide or the protein, or the protein activity in target cells or tissues, although various levels of inhibition may be achieved. Inhibiting or decreasing expression is typically referred to as knockdown.

The level of expression and/or activity of the PMP22 protein (full-length and/or truncated forms) may be inhibited by an RNAi molecule as disclosed herein by at least about 40%, or about 50%, or about 60%, or about 70%, or about 80%, or about 90%, or about 95%, or about 99%, or 100% compared to the level of expression and/or activity without the RNAi molecule.

In some embodiments, an RNAi molecule of interest may be an antisense oligonucleotide construct. Anti-sense oligonucleotides, including anti-sense RNA molecules and anti-sense DNA molecules, would typically act to directly block the translation of the targeted mRNA by binding thereto and thus preventing protein translation or increasing mRNA degradation, thus decreasing the level of the targeted protein, and thus activity, in a cell.

Thus, the RNAi molecule of interest may be an RNA or DNA sequence that is complementary to a target gene mRNA molecule expressed within a host cell, or it may be a DNA sequence encoding an RNA oligonucleotide or sequence that is complementary to a target gene mRNA molecule expressed within the host cell.

RNA interference refers to the process of sequence-specific post-transcriptional gene silencing in animals mediated by short/small interfering RNAs (siRNA). During RNAi, long double stranded RNA (dsRNA) stimulates the activity of a ribonuclease III enzyme referred to as “dicer.” Dicer is involved in processing of the long dsRNA into siRNA, which are short pieces of dsRNA. Short interfering RNAs derived from dicer activity are typically about 21-23 nucleotides in length and comprise about 19 base pair duplexes. Short interfering RNAs bind to the Argonaute proteins, and one strand of the dsRNA is removed, leaving the remaining strand available to bind to messenger RNA target sequences according to the rules of base pairing. The RNAi response also features an endonuclease complex containing an siRNA, commonly referred to as an RNA-induced silencing complex (RISC), which mediates cleavage of single-stranded RNA having sequence homologous to the siRNA. Cleavage of the target RNA takes place in the middle of the region complementary to the guide sequence of the siRNA duplex.

An RNAi molecule as described herein may comprise an antisense nucleic acid sequence which targets an mRNA and/or DNA encoding PMP22 gene product and is capable of reducing the amount of PMP22 expression and/or activity in cells.

That is to say, the antisense nucleic acid sequence comprises a sequence that is at least partially complementary, in particular perfectly complementary, to a region of the sequence of said mRNA, said complementarity being sufficient to yield specific binding under intra-cellular conditions. As immediately apparent to the skilled in the art, by a sequence that is “perfectly complementary to” a second sequence is meant the reverse complement counterpart of the second sequence, either under the form of a DNA molecule or under the form of an RNA molecule. A sequence is “partially complementary to” a second sequence if there are one or more mismatches.

An RNAi molecule described herein may be a double-stranded polynucleotide molecule comprising self-complementary sense and antisense regions, wherein the antisense region comprises a nucleotide sequence that is complementary to the nucleotide sequence of a part of exon 5 of PMP22 or a portion thereof, and the sense region has a nucleotide sequence corresponding to said part of exon 5 of PMP22 nucleic acid sequence or a portion thereof.

An RNAi molecule can be assembled from two separate oligonucleotides, where one strand is the sense strand and the other is the antisense strand, wherein the antisense and sense strands are self-complementary.

The RNAi molecule can also be assembled from a single oligonucleotide having self-complementary sense and antisense regions linked by means of a nucleic acid based or non-nucleic acid-based linker.

The RNAi molecule can be a polynucleotide that can form a substantially symmetrical duplex, asymmetric duplex, hairpin, or asymmetric hairpin secondary structure.

The RNAi molecule can also comprise a single stranded polynucleotide having nucleotide sequence complementary to a part of exon 5 of PMP22 nucleotide sequence or a portion thereof, wherein the single stranded polynucleotide can further comprise a terminal phosphate group, such as a 5′,3′-diphosphate or a 5′-phosphate as discussed, for example, in Martinez et al., 2002, Cell, and Schwarz et al., 2002, Molecular Cell.

Methods for selecting an appropriate dsRNA or dsRNA-encoding vector are known in the art for genes whose sequence is known (e.g., see Tuschl et al., Genes Dev. 1999; Elbashir et al., Nature. 2001; Hannon, Nature. 2002; McManus et al., RNA. 2002; Brummelkamp et al., Science. 2002; U.S. Pat. Nos. 6,573,099; 6,506,559; WO 01/36646; WO 99/32619; and WO 01/68836).

Methods for determining whether an oligonucleotide is capable of reducing the expression and/or activity of PMP22 in cells are known to those skilled in the art. This can be performed for example by analyzing PMP22 RNA expression such as by RT-qPCR, in situ hybridization or by analyzing PMP22 protein expression such as by immunohistochemistry, Western blot, and by comparing PMP22 protein expression or PMP22 functional activity in the presence and in the absence of the oligonucleotide to be tested.

An RNAi may be selected from the group consisting of siRNA, miRNA, dsRNA, and shRNA.

In some embodiments, an RNAi molecule is a miRNA.

In some embodiments, an RNAi molecule is a shRNA.

Small interfering RNA (siRNA) is a class of double-stranded RNA non-coding RNA molecules, typically 20-27 base pairs in length, operating within the RNA interference (RNAi) pathway to reduce or inhibit the expression of specific genes with complementary nucleotide sequences by degrading mRNA after transcription, and therefore preventing translation.

“siRNA” as used herein refers to a nucleic acids molecule capable of RNA interference or “RNAi”, as disclosed, for example, in Bass, 2001, Nature; Elbashir et al., 2001, Nature; WO 00/44895; WO 01/36646; WO 99/32619; WO 00/01846; WO 01/29058; WO 99/07409; and WO 00/44914. As used herein, siRNA molecules need not be limited to those molecules containing only RNA but may further encompass chemically modified nucleotides and non-nucleotides having RNAi capacity or activity.

Certain requirements for siRNA length, structure, chemical composition, and sequence that are needed to mediate efficient RNAi activity. siRNA duplexes comprising 21 nucleotides are most active when containing two nucleotide 3′-overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J.). Other studies have indicated that a 5′-phosphate on the target-complementary strand of a siRNA duplex is required for siRNA activity and that ATP is utilized in cells to maintain the 5′-phosphate moiety on the siRNA (Nykanen et al., 2001, Cell).

A microRNA (miRNA) is a small single-stranded non-coding RNA molecule (containing about 22 nucleotides) that functions in RNA silencing and post-transcriptional regulation of gene expression. miRNAs function via base-pairing with complementary sequences within mRNA molecules resulting in silencing the mRNA molecules. miRNAs resemble the small interfering RNAs (siRNAs) of the RNA interference (RNAi) pathway, except miRNAs derive from regions of RNA transcripts that fold back on themselves to form short hairpins, whereas siRNAs derive from longer regions of double-stranded RNA. miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem. The processed miRNA (also referred to as “mature miRNA”) becomes part of a large complex to downregulate, e.g., decrease translation, of a particular target gene.

A short hairpin RNA or small hairpin RNA (shRNA/Hairpin Vector) is an artificial RNA molecule with a tight hairpin turn that can be used to silence target gene expression via RNA interference (RNAi). Expression of shRNA in cells is typically accomplished by delivery of plasmids or through viral or bacterial vectors. shRNA may generally be expressed using a vector introduced into cells, wherein the vector utilizes the U6 promoter to ensure that the shRNA is always expressed. The shRNA hairpin structure is cleaved by the cellular machinery into siRNA.

In some embodiments, an RNAi molecule of the present invention may comprise a nucleic acid sequence having a length of at least 15 nucleotides.

In some embodiments, an RNAi molecule may comprise a nucleic acid sequence having a length from 15 to 25 nucleotides. For example, an RNAi molecule may have a length of 15, 16, 17, 18, 19, 20, 21, 22, 23, 24 or 25 nucleotides.

In one embodiment, an RNAi molecule may comprise a nucleic acid sequence having a length of about 21 nucleotides.

An RNAi molecule as disclosed herein may comprise a nucleic acid sequence having:
a ΔH ranging from about −138.4 to about −148.2 kcal/mol,
a ΔS ranging from about −0.39 to about −0.37 kcal/° K/mol,
a Tm ranging from about 49.58 to about 56.34° C., or ranging from about 52.16 to about 55.29° C.,
a GC content ranging from about 42.86 to about 52.38%, or from about 42.86 to about 47.62%
a ΔG ranging from about −32.88 to about −29.19 kcal/mol, or from about −32.54 to about −30.85 kcal/mol, or form about −32.16 to about −30.85 kcal/mol,
a 3′-end stability ranging from about −11.16 to about −6.58 kcal/mol, or from about −11.16 to about −6.7 kcal/mol, or from about −11.16 to about −6.73 kcal/mol, or from about −11.16 to about −7.71 kcal/mol, and/or
a 5′-end ΔG ranging from about −11.4 to about −5.73 kcal/mol, or from about −7.07 to about −6.22, or from about −6.72 to about −6.22 kcal/mol.

Melting Temperature (Tₘ): The melting temperature is calculated using the formula based on the nearest neighbor thermodynamic theory. It is the temperature at which half of the oligonucleotides are bonded. The formula is from the paper by Freier et al. (1986) Proc. Natl. Acad. Sci. U.S.A. 83, 9373-9377. These are the latest and most accurate nearest neighbor-based Tm calculations. Tm=ΔH/(ΔS+R*ln(C/4))+16.6 log ([K+]/(1+0.7 [K+]))−273.15 ΔH is enthalpy for helix formation. ΔS is entropy for helix formation. R is molar gas constant (1.987 cal/° C.*mol) C is the nucleic acids concentration. [K+] is salt concentration. Example: Say the primer sequence is ATCGATACGTAG (SEQ ID NO: 81). The ΔH and ΔS values of this primer will be 6-85000 cal/mol and −234.7 cal/° K/mol respectively (as calculated below). After substituting all the values, the Tm value of this primer will be 16.69° K.

GC %: GC % is the percentage of G and C in the primer. It is calculated by dividing the sum of G and C with the total number of bases present in the primer. Example: Say the primer sequence is ATCGATACGTAG (SEQ ID NO: 81). The GC % of this primer will be (5/12*100)=41.67%.

ΔG: This is the free energy of the primer calculated using the nearest neighbor method of Breslauer et al. (1986) Proc. Natl. Acad. Sci. U.S.A. 83, 3746-3750. ΔG is calculated by the formula ΔG=ΔH−TΔS. Here ΔH is the enthalpy of primer, T is the temperature, ΔS is the entropy of primer. T is set by ΔG temp. in the preferences. First the ΔH and ΔS are calculated and then the ΔG is calculated using their values. Example: Say the primer sequence is ATCGATACGTAG (SEQ ID NO: 81). Its ΔH and ΔS will be −85000 cal/mol and 7-234.7 cal/° K/mol respectively (as calculated below). Its ΔG will be −85000−(298.15*−234.7)=−15024.195 cal/mol=−15.02 kcal/mol.

3′ end stability: The stability of the primer determines its false priming efficiency. An ideal primer has a stable 5′ end and an unstable 3′ end. If the primer has a stable 3′ end, it will bond to a site which is complementary to it other than the target with its 5′ end hanging off the edge. It may then lead to secondary bands. Primers with low stability at the 3′ ends function well because the 3′ end bonding to false priming sites are too unstable to extend. The 3′ end stability is the ΔG value of the 5 bases of primer taken from 3′ end. The lower this value, numerically, the more liable the primer is to show secondary bands. Example: Say the primer sequence is ATCGATACGTAG (SEQ ID NO: 81). Its 3′ end stability will be ΔG(CGTAG)=ΔH(CGTAG)−298.15*ΔS(CGTAG). The ΔH and ΔS will be −32200 cal/mol and −82.8 cal/° K/mol resp. Thus its 3′ end stability will be −32200−(298.15*−82.8)=−7513.18 cal/mol=−7.51 kcal/mol.

ΔH: This is the enthalpy of the primer as calculated by the nearest neighbor method of Breslauer et al. (1986) Proc. Natl. Acad. Sci. U.S.A. 83, 3746-3750. ΔH for a pentamer is calculated as follows: ΔHATGCA=ΔHAT+ΔHTG+ΔHGC+ΔHCA. Example: Say the primer sequence is ATCGATACGTAG (SEQ ID NO: 81). Its ΔH will be (8600+5600+11900+5600+8600+6000+6500+11900+6500+6000+7800)=−85000 cal/mol=−85 kcal/mol.

ΔS: This is the entropy of the primer as calculated by the nearest neighbor method of Breslauer et al. (1986) Proc. Natl. Acad. Sci. U.S.A. 83, 3746-3750. ΔS for a pentamer is calculated as follows: ΔSATGCA=ΔSAT+ΔSTG+ΔSGC+ΔSCA. An initiation value of 15.1 is added to the ΔS calculation. Example: Say the primer sequence is ATCGATACGTAG (SEQ ID NO: 81). Its ΔS will be (23.9+13.5+27.8+13.5+23.9+16.9+17.3+27.8+17.3+16.9+20.8)+15.1=−234.7 cal/° K/mol=−0.23 kcal/° K/mol.

5′-end ΔG: Stability of the 5′ termini allows for efficient bonding of the primer to the target site. This stable 5′ region is called the GC Clamp. It ensures adequate binding of the primer to the template. Use of primers with optimal stability allows for the use of lower annealing temperatures without the production of secondary bands. Notice that the 3′ end should not be very stable and the 5′ end should have a strong GC clamp. The GC Clamp is the ΔG value of the 5 bases of primer taken from 5′ end. The lower this value, numerically, the more efficient is the primer. Example: Say the primer sequence is ATCGATACGTAG (SEQ ID NO: 81). Its 5′ ΔG will be ΔG(ATCGA)=ΔH(ATCGA)−298.15*ΔS(ATCGA).

In some embodiments, a ΔH may range from about −146.5 to about −144.5 kcal/mol.

In some embodiments, a ΔS may be about −0.38 kcal/° K/mol.

In some embodiments, a Tm may range from about 52.16 to about 54.69° C.

In some embodiments, a GC content may range from about 42.86 to about 47.62%.

In some embodiments, a ΔG may range from about −32.04 to about −30.85 kcal/mol.

In some embodiments, a 3′-end stability may range from about −11.16 to about −9.98 kcal/mol.

In some embodiments, a 5′-end ΔG may range from about −6.59 to about −6.22 kcal/mol.

In some embodiments, an RNAi molecule may comprise a nucleic acid sequence having a ΔH ranging from about −146.5 to about −144.5 kcal/mol, a ΔS of about −0.38 kcal/° K/mol, a Tm ranging from about 52.16 to about 54.69° C., a GC content ranging from about 42.86 to about 47.62%, a ΔG ranging from about −32.04 to about −30.85 kcal/mol, a 3′-end stability ranging from about −11.16 to about −9.98 kcal/mol, and/or a 5′-end ΔG ranging from about −6.22 to about −6.59 kcal/mol.

In one embodiment, an RNAi molecule may comprise a nucleic acid sequence having about 21 nucleotides, a ΔH ranging from about −148.2 to about −138.4 kcal/mol, and a ΔS ranging from about −0.39 to about −0.37 kcal/° K/mol.

In one embodiment, an RNAi molecule may comprise a nucleic acid sequence having about 21 nucleotides and a ΔH ranging from about −146.5 to about −144.5 kcal/mol.

In one embodiment, an RNAi molecule may comprise a nucleic acid sequence having about 21 nucleotides and a ΔS ranging of about −0.38 kcal/° K/mol.

In some embodiments, an RNAi molecule may target exon 5 of PMP22, may comprise a nucleic acid sequence having a length of 21 nucleotides, and may have a ΔH ranging from about 138.4 to about 148.2 kcal/mol.

In some embodiments, an RNAi molecule may target exon 5 of PMP22, may comprise a nucleic acid sequence having a length of 21 nucleotides, and may have a ΔH ranging from about −146.5 to about −144.5 kcal/mol.

In some embodiments, an RNAi molecule may target exon 5 of PMP22, may comprise a nucleic acid sequence having a length of 21 nucleotides, and may have a ΔS ranging of about 0.38 kcal/° K/mol.

An RNAi molecule as described herein may comprise a single stranded hairpin structure of about 36 to about 70 nucleotides in length, having two complementary sequences of about 15 to about 30 nucleotides separated by a spacer sequence that allows hybridization of the complementary sequences. Thus, the single stranded hairpin structure may have about 15 to about 30 bases pairs comprising the duplex portion of the molecule. In one embodiment, the hairpin siRNA may have about 18, 19, 20, or 21 base pairs in the duplex portion and a loop portion of a length that accommodates hybridization of the complementary RNAi sequences.

An RNAi molecule as disclosed herein may comprises an antisense nucleic acid sequence selected in the group consisting of SEQ ID NO: 55; SEQ ID NO: 56; SEQ ID NO: 60; SEQ ID NO: 61; SEQ ID NO: 62 and SEQ ID NO: 66, in particular in the group consisting in SEQ ID NO: 60 and SEQ ID NO: 61; and in particular said RNAi comprises a nucleic acid sequence of SEQ ID NO: 61.

An RNAi molecule as disclosed herein may be a dsRNA comprising a nucleic acid sequence selected in the group consisting of SEQ ID NO: 55; SEQ ID NO: 56; SEQ ID NO: 60; SEQ ID NO: 61; SEQ ID NO: 62 and SEQ ID NO: 66, as antisense strand and a nucleic acid sequence selected in the group consisting of SEQ ID NO: 11; SEQ ID NO: 12; SEQ ID NO: 16; SEQ ID NO: 17; SEQ ID NO: 18; and SEQ ID NO: 22, as sense strand.

An RNAi molecule may be a shRNA comprising a nucleic acid sequence selected in the group consisting of SEQ ID NO: 55; SEQ ID NO: 56; SEQ ID NO: 60; SEQ ID NO: 61; SEQ ID NO: 62 and SEQ ID NO: 66, as antisense strand and a nucleic acid sequence selected in the group consisting of SEQ ID NO: 11; SEQ ID NO: 12; SEQ ID NO: 16; SEQ ID NO: 17; SEQ ID NO: 18; and SEQ ID NO: 22, as sense strand.

An RNAi molecule may be a siRNA comprising a nucleic acid sequence selected in the group consisting of SEQ ID NO: 55; SEQ ID NO: 56; SEQ ID NO: 60; SEQ ID NO: 61; SEQ ID NO: 62 and SEQ ID NO: 66, as antisense strand and a nucleic acid sequence selected in the group consisting of SEQ ID NO: 11; SEQ ID NO: 12; SEQ ID NO: 16; SEQ ID NO: 17; SEQ ID NO: 18; and SEQ ID NO: 22, as sense strand.

In one embodiment, an RNAi molecule of the invention RNAi does not comprise a nucleic acid sequence antisense selected in the group consisting of SEQ ID NO: 57; SEQ ID NO: 58; SEQ ID NO: 59; SEQ ID NO: 63; and SEQ ID NO: 64.

In some embodiments, an RNAi molecule as disclosed herein may comprises an antisense nucleic acid sequence selected in the group consisting of SEQ ID NO: 60 and SEQ ID NO: 61. An RNAi molecule as disclosed herein may comprise an antisense nucleic acid sequence of SEQ ID NO: 61.

In some embodiments, an RNAi molecule as disclosed herein may be a dsRNA comprising a nucleic acid sequence selected in the group consisting of SEQ ID NO: 60 and SEQ ID NO: 61, as antisense strand and a nucleic acid sequence selected in the group consisting of SEQ ID NO: 16 and SEQ ID NO: 17, as sense strand.

An RNAi molecule as disclosed herein may be a dsRNA comprising a nucleic acid sequence of SEQ ID NO: 61 as antisense strand and a nucleic acid sequence of SEQ ID NO: 17, as sense strand.

Nucleic acid sequences as disclosed herein may comprise chemically modified nucleotides or a modified backbone structure, e.g., a backbone other than the standard phosphodiester linkage found in natural nucleic acid sequence or may be conjugated. Modifications may be stabilizing modifications improving the resistance to in vivo degradation and therefore the efficacy of the nucleic acid sequence.

An RNAi molecule as disclosed herein may comprise at least one modified nucleotide.

Modified nucleic acid sequence or nucleic acid sequence analog support bases capable of hydrogen bonding by Watson-Crick base pairing to standard polynucleotide bases, where the modified backbone presents the bases in a manner to permit such hydrogen bonding in a sequence-specific fashion between the oligonucleotide analog molecule and bases in a standard polynucleotide (e.g., single-stranded RNA or single-stranded DNA).

A modified backbone structure includes linkages such as phosphate, phosphorothioate, phosphorodithioate, phosphoroselenoate, phosphorodiselenoate, phosphoroanilothioate, phoshoraniladate, phosphoroamidate, and the like.

Modified nucleotides may include chemical modifications occurring at the phosphate groups or on the sugar moiety.

The term “nucleic acid sequence” also refers to a nucleic acid sequence that is inverted relative to its normal orientation for transcription and so correspond to an RNA or DNA sequence that is complementary to a target gene mRNA molecule expressed within the host cell (e.g., it can hybridize to the target gene mRNA molecule through Watson-Crick base pairing). An antisense strand can be constructed in a number of different ways, provided that it is capable of interfering with the expression of a target gene. For example, the antisense strand can be constructed by reverse-complementing the coding region (or a portion thereof) of the target gene relative to its normal orientation for transcription to allow the transcription of its complement, (e.g., RNAs encoded by the antisense and sense gene may be complementary). In some embodiments, the oligonucleotide need not to have the same intron or exon pattern as the target gene, and noncoding segments of the target gene may be equally effective in achieving antisense suppression of target gene expression as coding segments such as antisense oligonucleotide (ASO). In some embodiments, the oligonucleotide has the same exon pattern as the target gene such as siRNA and antisense oligonucleotide (ASO).

Chemically modified oligonucleotides by backbone modifications include morpholinos, phosphorodiamidate morpholino oligomers (Phosphorodiamidate morpholinos, PMO), peptide nucleic acid (PNA), phosphorothioate (PS) oligonucleotides, stereochemically pure phosphorothioate (PS) oligonucleotides, phosphoramidates modified oligonucleotides, thiophosphoramidate-modified oligonucleotides, and methylphosphonate modified oligonucleotides; chemically modified oligonucleotide by heterocycle modifications such as bicycle modified oligonucleotides, Bicyclic Nucleic Acid (BNA), tricycle modified oligonucleotides, tricyclo-DNA-antisense oligonucleotides (ASOs), nucleobase modifications such as 5-methyl substitution on pyrimidine nucleobases, 5-substituted pyrimidine analogs, 2-Thio-thymine modified oligonucleotides, and purine modified oligonucleotides; chemically modified oligonucleotide by sugar modifications such as Locked Nucleic Acid (LNA) oligonucleotides, 2′,4′-Methyleneoxy Bridged Nucleic Acid (BNA), ethylene-bridged nucleic acid (ENA), constrained ethyl (cEt) oligonucleotides, 2′-Modified RNA, 2′- and 4′-modified oligonucleotides such as 2′-O-Me RNA (2′-OMe), 2′-O-Methoxyethyl RNA (MOE), 2′-Fluoro RNA (FRNA), and 4′-Thio-Modified DNA and RNA.

Chemically modified oligonucleotide by conjugation strategies such as N-acetyl galactosamine (GalNAc) oligonucleotide conjugates such as 5′-GalNAc and 3′-GalNAc ASO conjugates, lipid oligonucleotide conjugates (LASO), cell penetrating peptides (CPP) oligonucleotide conjugates, targeted oligonucleotide conjugates, antibody-oligonucleotide conjugates, polymer-oligonucleotide conjugate such as with PEGylation and targeting ligand.

Stabilization can be accomplished via phosphate backbone modifications, phosphodiester modifications, phosphorothioate (PS) backbone modifications, combinations of phosphodiester and phosphorothioate modifications, thiophosphoramidate modifications, 2′ modifications (2′-50-Me, 2′-O-(2-methoxyethyl) (MOE) modifications and 2′-fluoro modifications), methylphosphonate, methylphosphorothioate, phosphorodithioate, p-ethoxy, and combinations.

In some embodiments, the oligonucleotide used in the context of the invention comprises modified nucleotides selected from the group consisting of LNA, 2′-OMe analogs, 2′-O-Met, 2′-O-(2-methoxyethyl) (MOE) oligomers, 2′-phosphorothioate analogs, 2′-fluoro analogs, 2′-Cl analogs, 2′-Br analogs, 2′-CN analogs, 2′-CF3 analogs, 2′-OCF3 analogs, 2′-OCN analogs, 2′-O-alkyl analogs, 2′-S-alkyl analogs, 2′-N-alkyl analogs, 2′-O-alkenyl analogs, 2′-S-alkenyl analogs, 2′-N-alkenyl analogs, 2′-SOCH3 analogs, 2′-SO2CH3 analogs, 2′-ONO2 analogs, 2′-NO2 analogs, 2′-N3 analogs, 2′-NH2 analogs, tricyclo (tc)-DNAs, U7 short nuclear (sn) RNAs, tricyclo-DNA-oligoantisense molecules and combinations thereof.

In some embodiments, an RNAi molecule as disclosed herein may be conjugated with different compounds to enhance cell delivery.

An RNAi molecule may be conjugated to a compound that may assist in cell delivery, which may be targeting agents such as antibodies or GalNAc (N-acetylgalactosamine).

An RNAi molecule may be conjugated to a compound increasing the stability, the half-life or the delivering capacity of the RNAi molecules. Suitable compounds may cationic polymers, e.g., polyethylenimines or cationic peptides, such as poly(L-lysines) or protamines, or lipid compounds, such as squalene.

Conjugation of an RNAi molecule as disclosed herein to a suitable compound may be carried out by any techniques known in the art.

For example, an RNAi as disclosed herein may be conjugated with squalene molecule as disclosed in Boutary et al. Commun Biol. 2021 Mar. 9; 4(1):317 or in WO 2020/064749.

Typically, the nucleic acid sequence disclosed herein may be obtained by conventional methods well known to those skilled in the art. For example, the oligonucleotide of the invention can be synthesized de novo using any of a number of procedures well known in the art, for example, by the b-cyanoethyl phosphoramidite method. These chemistries can be performed by a variety of automated nucleic acids synthesizers available in the market. These nucleic acids may be referred to as synthetic nucleic acids. Alternatively, oligonucleotide can be produced on a large scale in plasmids. Oligonucleotide can be prepared from existing nucleic acid sequences using known techniques, such as those employing restriction enzymes, exonucleases or endonucleases. Oligonucleotide prepared in this manner may be referred to as isolated nucleic acids.

The one skilled in the art can easily provide some approaches and modifications for enhancing the delivery and the efficacy of oligonucleotides such as chemical modification of the oligonucleotides, lipid- and polymer-based nanoparticles or nanocarriers, ligand-oligonucleotide conjugates by linking oligonucleotides to targeting agents such as carbohydrates, peptides, antibodies, aptamers, lipids or small molecules and small molecules that improve oligonucleotide delivery. Lipophilic conjugates and lipid conjugates include fatty acid-oligonucleotide conjugates; sterol-oligonucleotide conjugates and vitamin-oligonucleotide conjugates.

A delivery system, or a delivery nucleic acid sequence system, in accordance with the invention may comprise a nucleic acid sequence consisting in or coding for an RNAi molecule as disclosed herein.

A vector transports the nucleic acid sequence to cells with reduced degradation relative to the extent of degradation that would result in the absence of the vector.

A delivery system comprising a nucleic acid sequence consisting in or coding for an RNAi molecule may be a non-viral delivery system, such as, for example, a naked recombinant DNA molecule; a naked recombinant RNA molecule; a plasmid; a phagemid; optionally formulated with a delivery agents, such as cationic transfection agents, liposomes, lipid nanoparticles, niosomes, and the like; or a viral delivery system, such as an adeno-associated virus, an adenovirus, a retrovirus, an herpes simplex virus, a vaccinia virus, an SV40-type virus, a polyoma virus, an Epstein-Barr virus, a papilloma virus, a lentivirus, or a poliovirus.

A delivery system may comprise an expression vector.

An expression vector may be an isolated expression vector.

An expression vector may be a recombinant expression vector.

Typically, an expression vector of the present invention may comprise an expression cassette. The term “expression cassette”, as used herein, refers to a nucleic acids construct comprising nucleic acids elements sufficient for the expression of the polynucleotide of interest. Typically, an expression cassette comprises a nucleic acid sequence coding for an RNAi molecule of the invention operatively linked to a promoter. As used herein, the term “promoter”, as used herein, refers to a nucleic acid sequence capable of controlling the expression of a coding sequence or functional RNA. The term “operatively linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

Promoter suitable for the invention may include promoters derived from the genome of mammalian cells or from viruses, for example mammalian viruses. Encoding sequences can be operatively linked to regulatory sequences in sense or antisense orientation. In some embodiments, the promoter is a heterologous promoter. The term “heterologous promoter”, as used herein, refers to a promoter that does is not found to be operatively linked to a given encoding sequence in nature. In some embodiments, an expression cassette may comprise additional elements, for example, an intron, an enhancer, a polyadenylation site, a woodchuck response element (WRE), and/or other elements known to affect expression levels of the encoding sequence. In general, the polynucleotide of interest is located 3′ of a promoter sequence.

In some embodiments, a promoter sequence consists of proximal and more distal upstream elements and can comprise an enhancer element. An “enhancer” is a nucleic acid sequence that can stimulate promoter activity and may be an innate element of the promoter or a heterologous element inserted to enhance the level or tissue-specificity of a promoter. In some embodiments, the promoter is derived in its entirety from a native gene. In some embodiments, the promoter is composed of different elements derived from different naturally occurring promoters. In some embodiments, the promoter comprises a synthetic nucleic acid sequence. It will be understood by those skilled in the art that different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g., tetracycline-responsive promoters) are well known to those of skill in the art.

According to the present invention, the promoter linked to a nucleic acid sequence encoding an RNAi molecule of the invention is operable in, for example, animal cells, such as mammalian cells, to control expression of the RNAi molecule. In some embodiments, the promoter is operable in human cells.

A promoter can be of human origin or from other species, including from mice. In addition, sequences derived from nonviral genes, such as the murine metallothionein gene, will also find use herein. Such promoter sequences are commercially available from, e.g., Stratagene (San Diego, CA).

Examples of promoter include, but are not limited to, the phosphoglycerate kinase (PKG) promoter, CAG, NSE (neuronal specific enolase), synapsin or NeuN promoters, the SV40 early promoter, a vaccinia virus 7.5K promoter, mouse mammary tumor virus LTR promoter; adenovirus major late promoter (Ad MLP); an herpes simplex virus (HSV) promoter, a cytomegalovirus (CMV) promoter such as the CMV immediate early promoter region (CMVIE), SFFV promoter, Rous sarcoma virus (RSV) promoter, synthetic promoters, hybrid promoters, an EF1 alpha promoter, a metallothionein promoter, a beta-actin promoter, a human IL-2 gene promoter, a human IFN gene promoter, a human IL-4 gene promoter, a human lymphotoxin gene promoter, or human GM-CSF gene promoter, MPZ gene promoter and the like, CX32 gene promoter and the like, MBP promoter and the like, CNPase promoter and the like, a RNA polymerase III promoter, such as a U6 promoter, H1 promoter and the like.

In one embodiment, a promoter may be an RNA polymerase III promoter. In another embodiment, a promoter may be a U6 or H1 promoter.

In some embodiments, a delivery system may be a viral delivery system. A viral delivery system may comprise a viral expression vector.

A viral expression vector may be selected in the group consisting in adeno-associated virus, adenovirus, retrovirus, herpes simplex virus, vaccinia virus, SV40-type virus, polyoma virus, Epstein-Barr virus, papilloma virus, lentivirus, and poliovirus.

In some embodiments, an expression vector may be an adeno-associated virus.

In some embodiments, the AAV vector is AAV1, AAV2, AAV3, AAV4, AA5, AAV6, AAV7, AAV8, AAV9, AAV10, AAVrh10, AAV2/9, AAV2/rh10 or any other serotypes of AAV that can infect human, rodents, monkeys or other species.

The AAV vector may be a single-stranded AAV or a self-complementary AAV.

In some embodiments the AAV vector may be recombinant adeno-associated virus (rAAV). The rAAV may be of any serotype, modification, or derivative, known in the art, or any combination thereof (e.g., a population of rAAV that comprises two or more serotypes, e.g., comprising two or more of rAAV2, rAAV8, and rAAV9) known in the art. In some embodiments, the rAAV are rAAV1, rAAV2, rAAV3, rAAV4, rAAV5, rAAV6, rAAV7, rAAV8, rAAV9, rAAV10, rAAV-11, rAAV-12, rAAV-13, rAAV-14, rAAV-15, rAAV-16, rAAV.rh8, rAAV.rh10, rAAV.rh20, rAAV.rh39, rAAV.Rh74, rAAV.RHM4-1, AAV.hu37, rAAV.Anc80, rAAV.Anc80L65, rAAV.7m8, rAAV.PHP.B, rAAV2.5, rAAV2tYF, rAAV3B, rAAV.LK03, rAAV.HSC1, rAAV.HSC2, rAAV.HSC3, rAAV.HSC4, rAAV.HSC5, rAAV.HSC6, rAAV.HSC7, rAAV.HSC8, rAAV.HSC9, rAAV.HSC10, rAAV.HSC11, rAAV.HSC12, rAAV.HSC13, rAAV.HSC14, rAAV.HSC15, or rAAV.HSC16, or other rAAV, or combinations of two or more thereof.

In some embodiments, an adeno-associated virus may be AAV9, AAV2/9, or AAVrh10.

As used herein the term “AAV9 vector” has its general meanings in the art and refers to a vector derived from an adeno-associated virus serotype 9. In particular, the term “AAV9”, as used herein, refers to a serotype of adeno-associated virus with a genome sequence as defined in the GenBank accession number AAS99264. The AAV9 vector of the present invention can have one or more of the AAV9 wild-type genes deleted in whole or part, preferably the rep and/or cap genes, but retain functional flanking ITR sequences.

An adeno-associated virus vector can have one or more of the wild-type genes deleted in whole or part, preferably the rep and/or cap genes, but retain functional flanking ITR sequences. Functional ITR sequences are necessary for the rescue, replication and packaging of the AAV virion. Thus, an adeno-associated virus vector is defined herein to include at least those sequences required in cis for replication and packaging (e. g., functional ITRs) of the virus. The ITRs need not be the wild-type nucleic acid sequences, and may be altered, e.g., by the insertion, deletion or substitution of nucleotides, so long as the sequences provide for functional rescue, replication and packaging.

Adeno-associated virus expression vectors are constructed using known techniques to at least provide as operatively linked components in the direction of transcription, control elements including a transcriptional initiation region, the polynucleotide of interest and a transcriptional termination region.

The control elements are selected to be functional in a mammalian cell. The resulting construct which contains the operatively linked components is bounded (5′ and 3′) with functional ITR sequences. By “adeno-associated virus inverted terminal repeats” or “ITRs” is meant the art-recognized regions found at each end of the AAV genome which function together in cis as origins of DNA replication and as packaging signals for the virus. ITRs, together with the rep coding region, provide for the efficient excision and rescue from, and integration of a nucleic acid sequence interposed between two flanking ITRs into a mammalian cell genome.

In some embodiments, in adeno-associated virus expression vectors, a promoter operably linked to a nucleic acid sequence encoding an RNAi molecule may be an RNA polymerase III promoter. In one embodiment, an RNA polymerase III promoter may be a U6 promoter.

The adeno-associated virus (AAV) vector of the present invention can be constructed by directly inserting the nucleic acid sequence of interest into an adeno-associated virus genome which has had the major adeno-associated virus open reading frames (“ORFs”) excised therefrom. Other portions of the adeno-associated virus genome can also be deleted, so long as a sufficient portion of the ITRs remain to allow for replication and packaging functions. Such constructs can be designed using techniques well known in the art. (See, e. g. U.S. Pat. Nos. 5,173,414; 5,139,941; WO 92/01070; 93/03769; Lebkowski et al., Mol Cell Biol. 1988; Carter, Curr Opin Biotechnol. 1992; Muzyczka, Cuff Top Microbiol Immunol. 1992; Kotin; Hum Gene Ther. 1994; Shelling and Smith, Gene Ther. 1994; and Zhou et al., J Exp Med. 1994). Alternatively, AAV ITRs can be excised from the viral genome or from an AAV vector containing the same and fused 5′ and 3′ of a selected nucleic acids construct that is present in another vector using standard ligation techniques. Adeno-associated virus vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226. Additionally, chimeric genes can be produced synthetically to include AAV ITR sequences arranged 5′ and 3′ of one or more selected nucleic acid sequences. Preferred codons for expression of the chimeric gene sequence in mammalian PNS cells can be used. The complete chimeric sequence is assembled from overlapping oligonucleotides prepared by standard methods.

An AAV as disclosed herein may be made by co-transfecting a plasmid containing a nucleic acid sequence encoding an RNAi molecule flanked by the two AAV terminal repeats (McLaughlin et al., J Virol. 1988; Srivastava et al., Proc Natl Acad Sci USA. 1989) and an expression plasmid containing the wild type AAV coding sequences without the terminal repeats (McCarty et al., J. Virol. 1991).

In order to produce AAV virions, an AAV expression vector may be introduced into a suitable host cell using known techniques, such as by transfection. A number of transfection techniques are generally known in the art. See, e. g., Sambrook et al., 2001, MOLECULAR CLONING: A LABORATORY MANUAL, 3d ed., Cold Spring Harbor Laboratory Press, Cold Spring Harbor, N.Y. Basic Methods in Molecular Biology, Elsevier. Particularly suitable transfection methods include calcium phosphate co-precipitation, direct microinjection into cultured cells, electroporation, liposome mediated gene transfer, lipid-mediated transduction, or nucleic acids delivery using high-velocity microprojectiles.

In some embodiments, the invention relates to an adeno-associated virus (AAV) vector comprising an RNAi molecule comprising an antisense nucleic acid sequence that inhibits or reduces the expression of the PMP22 protein.

In some embodiments, the invention relates to an adeno-associated virus (AAV) vector comprising an RNAi molecule comprising a miRNA sequence that inhibits or reduces the expression of the PMP22 protein.

In some embodiments, the invention relates to an adeno-associated virus (AAV) vector comprising an RNAi molecule comprising a shRNA sequence that inhibits or reduces the expression of the PMP22 protein.

In some embodiments, the invention relates to an adeno-associated virus (AAV) vector comprising an RNAi molecule comprising an antisense nucleic acid sequence which targets a region comprising or consisting in the nucleic acid sequence ranging from position 638 to position 690 of SEQ ID NO: 79 (mRNA transcript: NCBI Reference Sequence: NM_000304.4).

In some embodiments, the invention relates to an adeno-associated virus (AAV) vector comprising an RNAi molecule comprising an antisense nucleic acid sequence selected in the group consisting of SEQ ID NO: 55; SEQ ID NO: 56; SEQ ID NO: 60; SEQ ID NO: 61; SEQ ID NO: 62 and SEQ ID NO: 66.

In some embodiments, the invention relates to an adeno-associated virus (AAV) vector comprising an RNAi molecule comprising an antisense nucleic acid sequence selected in the group consisting in SEQ ID NO: 60 and SEQ ID NO: 61.

In some embodiments, the invention relates to an adeno-associated virus (AAV) vector comprising an RNAi molecule comprising an antisense nucleic acid sequence of SEQ ID NO: 61.

In some embodiments, the invention relates to an adeno-associated virus (AAV) vector comprising an RNAi molecule comprising, or consisting of, an antisense oligonucleotide of sequence SEQ ID NO: 61, and/or an oligonucleotide coding for an antisense oligonucleotide of sequence SEQ ID NO: 61.

In some embodiments, the invention relates to an adeno-associated virus (AAV) vector comprising an RNAi molecule comprising, or consisting of, an antisense oligonucleotide having the sequence of SEQ ID NO: 61, and/or an oligonucleotide coding for an antisense oligonucleotide having the sequence of SEQ ID NO: 61.

In some embodiments, the invention relates to an adeno-associated virus (AAV) vector comprising an RNAi molecule being an antisense oligonucleotide having the sequence of SEQ ID NO: 61, and/or an oligonucleotide coding for an antisense oligonucleotide having the sequence of SEQ ID NO: 61.

In some embodiments, the invention relates to an adeno-associated virus (AAV) vector comprising an RNAi molecule comprising, or consisting of, at least one antisense oligonucleotide, wherein the sequence of said at least one antisense oligonucleotide consists of the sequence SEQ ID NO: 61.

In some embodiments, the invention relates to an adeno-associated virus (AAV) vector comprising an RNAi molecule comprising, or consisting of, at least one antisense nucleic acid sequence, wherein said antisense nucleic acid sequence consists of the sequence SEQ ID NO: 61.

In some embodiments, the invention relates to an adeno-associated virus (AAV) vector comprising an RNAi molecule comprising, or consisting of, at least one antisense oligonucleotide coding sequence, wherein said at least one antisense oligonucleotide coding sequence consists of the sequence SEQ ID NO: 61.

In some embodiments, the invention relates to an adeno-associated virus (AAV) vector comprising an RNAi molecule comprising, or consisting of, at least one antisense nucleic acid coding sequence, wherein said antisense nucleic acid coding sequence consists of the sequence SEQ ID NO: 61.

In some embodiments, the invention relates to an adeno-associated virus (AAV) vector comprising an RNAi molecule comprising, or consisting of, a sequence encoding an antisense oligonucleotide, wherein said sequence encoding an antisense oligonucleotide consists of the sequence SEQ ID NO: 61.

In some embodiments, the invention relates to an adeno-associated virus (AAV) vector comprising an RNAi molecule comprising, or consisting of, a sequence encoding an antisense nucleic acid sequence, wherein said sequence encoding an antisense nucleic acid sequence consists of the sequence SEQ ID NO: 61.

RNAi molecule delivery viral vectors useful in the practice of the present invention can be constructed utilizing methodologies well known in the art of molecular biology. Typically, viral vectors are assembled from nucleic acid sequence of the RNAi molecule, suitable regulatory elements and elements necessary for production of viral proteins which mediate cell transduction.

Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

The invention relates to an isolated host cell containing an RNAi molecule or a nucleic acid delivery system disclosed herein.

A host cell of the invention may be a recombinant host cell.

The term “host cell” is used to refer to a cell which has been transfected with a nucleic acid sequence of interest and then having it expressed, or a cell which has been transformed with a viral vector comprising a nucleic acid sequence of interest. The term includes the progeny of the parent cell, whether or not the progeny is identical in morphology or in genetic make-up to the original parent, so long as the gene is present.

The term “transfection” is used to refer to the uptake of foreign or exogenous DNA or RNA by a cell, and a cell has been “transfected” when the exogenous DNA or RNA has been introduced inside the cell. A number of transfection techniques are well known in the art and are disclosed herein. See, e.g., Sambrook et al., 2001, MOLECULAR CLONING: A LABORATORY MANUAL, 3d ed., Cold Spring Harbor Laboratory Press, Cold Spring Harbor, N.Y.; Davis et al., and 1986, BASIC METHODS IN MOLECULAR BIOLOGY (Elsevier).

The introduction of an expression vector comprising or encoding an RNAi molecule into a selected host cell or target cell may be accomplished by well-known methods including methods such as transfection, infection, calcium chloride, electroporation, microinjection, lipofection, DEAE-dextran method, or other known techniques as described above. The method selected will in part be a function of the type of host cell or target cell to be used. These methods and other suitable methods are well known to the skilled artisan, and are set forth, for example, in Sambrook et al., 2001, MOLECULAR CLONING: A LABORATORY MANUAL, 3d ed., Cold Spring Harbor Laboratory Press, Cold Spring Harbor, N.Y.

Where the present gene delivery system is constructed on the basis of viral vector construction, the contacting is performed as conventional infection methods known in the art. The infection of hosts using viral vectors is well described in the above-cited publications.

Where the nucleic acid sequence delivery system is a naked recombinant DNA molecule or plasmid, the nucleic acid sequence to be delivered may be introduced into cells by techniques known in the art, such as microinjection, calcium phosphate co-precipitation, electroporation, liposome-mediated transfection, DEAE-dextran treatment, and particle bombardment.

In some embodiments, a host cell may be a prokaryotic cell, such as a bacteria cell, or an eukaryotic cell, for example a yeast cell, a fungi cell, a plant cell or mammalian cell, such as a rodent, a non-human primate cell or a human cell.

In some embodiments, a host cell may be a HEK293 cell, such as a HEK293T cell.

In some embodiments, a host cell may be a nerve cell, for example a Schwann cell or a neuron.

A transfected host cell of the invention is expressing an RNAi molecule of interest as disclosed herein.

The host cell of the invention may comprise or contain any adeno-associated virus (AAV) vector comprising any RNAi molecule as described hereinabove in the specification.

In some embodiments, the host cell of the invention comprises or contains an adeno-associated virus (AAV) vector comprising an RNAi molecule comprising an antisense oligonucleotide of sequence SEQ ID NO: 61, and/or an oligonucleotide coding for an antisense oligonucleotide of sequence SEQ ID NO: 61.

An RNAi molecule or an expression vector, in particular an adeno-associated virus vector, as disclosed herein may be formulated into a pharmaceutical composition comprising at least one pharmaceutically acceptable excipient.

In a pharmaceutical composition as disclosed herein an RNAi molecule or an expression vector is an active ingredient.

A pharmaceutical composition as disclosed herein comprises an active ingredient in a therapeutically effective amount.

Dosage and dosing frequency will depend upon the pharmacokinetic parameters of the RNAi molecules disclosed herein. For example, a clinician administers the RNAi molecules until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

“Pharmaceutically” or “pharmaceutically acceptable” refer to molecular entities and compositions that do not produce an adverse, allergic or other untoward reaction when administered to a mammal, especially a human, as appropriate.

A pharmaceutically acceptable excipient may be a carrier, a buffer, a stabilizer or other materials well known to those skilled in the art. Such materials should be non-toxic and should not interfere with the efficacy of the active ingredient (i.e., the RNAi molecule or the expression vector of the invention). The precise nature of the carrier or other material may be determined by the skilled person according to the route of administration.

A pharmaceutical composition may contain formulation excipients for modifying, maintaining or preserving, for example, the pH, osmolarity, viscosity, clarity, color, isotonicity, odor, sterility, stability, rate of dissolution or release, adsorption or penetration of the composition. Suitable formulation excipients include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, REMINGTON's PHARMACEUTICAL SCIENCES, 18th Edition, (A. R. Gennaro, ed.), 1990, Mack Publishing Company.

Optimal pharmaceutical compositions can be determined by one skilled in the art depending upon, for example, the intended route of administration, delivery format and desired dosage. See, for example. REMINGTON's PHARMACEUTICAL SCIENCES, Id. Such compositions may influence the physical state, stability, rate of in vivo release and rate of in vivo clearance.

A pharmaceutical composition may be in liquid form or in solid form.

Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included.

An RNAi molecule, a nucleic acids delivery system, or a pharmaceutical composition as disclosed herein may be administered by systemic route or local route. Suitable systemic route may be selected in the group consisting in enteral or parenteral routes. Parenteral administration comprises intravenous, intraperitoneal, intraarterial, intra-articular, intra-lymphatic, subcutaneous, and intra-nerve route. Local route may be selected in the group consisting in intracerebral, intramuscular, intrathecal and intra-nerve (intraneural) routes.

Preferably, the vector or pharmaceutical composition of the invention is to be administered by a systemic, intrathecal or intraneural route.

The administration may be carried out by systemic route. Suitable systemic route may be selected in the group consisting in enteral, intravenous, intraperitoneal, intraarterial, intra-articular, intra-lymphatic, and subcutaneous.

The intra-nerve, or intraneural, injection may be a sciatic, a tibial, a fibular, a radial or a median nerve injection.

An RNAi molecule, a nucleic acids delivery system, or a pharmaceutical composition as disclosed herein may be administered local route, for example in a nerve, for example in the sciatic, tibial, fibular, radial and median nerves.

For injection, the active ingredient will be in the form of an aqueous solution, which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Vehicles for a formulation capable of being injected may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions.

In all cases, the form must be sterile and must be fluid to the extent that easy syringeability exists.

In certain embodiments, sterile composition may be obtained by filtration through sterile filtration membranes.

In certain embodiments, the composition may be a solid composition. A solid composition may be lyophilized composition. Where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

For solid compositions, conventional non-toxic solid carriers can be used, for example, pharmaceutical grades of mannitol, lactose, starch, magnesium stearate, sodium saccharin, talcum, cellulose, glucose, sucrose, magnesium carbonate, and the like.

For delayed release, the vector may be included in a pharmaceutical composition, which is formulated for slow release, such as in microcapsules formed from biocompatible polymers or in liposomal carrier systems according to methods known in the art.

In certain embodiments, the invention provides pharmaceutical compositions comprising a therapeutically effective amount of an RNAi molecule or an expression vector as disclosed herein, for example comprising an antisense nucleic acid sequence selected in the group consisting of SEQ ID NO: 55; SEQ ID NO: 56; SEQ ID NO: 60; SEQ ID NO: 61; SEQ ID NO: 62 and SEQ ID NO: 66, in particular in the group consisting in SEQ ID NO: 60 and SEQ ID NO: 61; and in particular said RNAi comprises a nucleic acid sequence of SEQ ID NO: 61, that inhibits PMP22 expression in mammalian cells, together with a pharmaceutically acceptable excipient.

The pharmaceutical composition of the invention may comprise any adeno-associated virus (AAV) vector comprising any RNAi molecule as described hereinabove in the specification, and a pharmaceutically acceptable excipient.

In certain embodiments, the invention provides pharmaceutical compositions comprising a therapeutically effective amount of an adeno-associated virus (AAV) vector comprising an RNAi molecule comprising an antisense oligonucleotide of sequence SEQ ID NO: 61, and/or an oligonucleotide coding for an antisense oligonucleotide of sequence SEQ ID NO: 61, together with a pharmaceutically acceptable excipient.

In some embodiments, the present invention provides an RNAi molecule or an expression vector as disclosed herein, as a medicament.

An RNAi molecule, an expression vector according, or a pharmaceutical composition as disclosed herein may be for use in preventing and/or treating a Charcot-Marie-Tooth type 1A or 1E diseases in a patient in need thereof.

An RNAi molecule, an expression vector, or a pharmaceutical composition may be administered in the sciatic nerve.

The invention also relates to the use of an RNAi molecule as disclosed herein, an expression vector containing the same, or a pharmaceutical composition containing at least one such RNAi molecule or expression vector for the prevention and/or treatment of CMT1A or 1E diseases.

Also, the invention relates to an RNAi molecule as disclosed herein, or an expression vector containing the same, for the manufacture of medicament for the prevention and/or treatment of CMT1A or 1E diseases.

In some embodiments, the present invention provides a method for preventing and/or treating a Charcot-Marie-Tooth type 1A or 1E diseases in a patient in need thereof, the method comprising at least a step of administering an RNAi molecule, an expression vector, or a pharmaceutical composition as disclosed herein to said patient.

The method may be carried out by intra-nerve injection. The intra-nerve injection may be a sciatic nerve injection. Suitable protocols for sciatic nerve injection are disclosed in WO 2017/005806.

An adequate administration amount of an RNAi molecule, an expression vector according, or a pharmaceutical composition as disclosed herein may vary depending on various factors including age, sex or disease condition of the patient, absorption rate of effective ingredients in body, elimination rate and combined drugs.

An RNAi molecule, an expression vector according, or a pharmaceutical composition may be administered in a daily dosage.

Administering an RNAi molecule, an expression vector according, or a pharmaceutical composition of the invention may be done by direct injection into the nerve.

An effective dose within the context of the invention may be a dose allowing an optimal transduction of the Schwann cells.

For an RNAi molecule formulated in a viral delivery system, such as an adeno-associated virus, typically, from 10¹⁰ to 10¹⁴ viral genomes (vg) may be administered in human.

By “treatment of Charcot-Marie-Tooth 1A or 1E diseases”, it is herein meant stopping, at least partially, the evolution of the disease or reversing the disease. Desirable effects of treatment for example may comprise: preventing or reducing weakness and/or atrophy of the muscles of the lower legs, hand weakness and/or sensory loss, thereby normalizing gait and/or preventing or reducing foot drops, stopping, slowing down or curing weakness and/or atrophy of the muscles of the lower legs, hand weakness and/or sensory loss, thereby normalizing gait and/or reducing foot drops, and/or normalizing the muscle conductive velocity.

In some embodiments, the invention relates to a method of selecting an RNA interferent (RNAi) molecule that inhibits a PMP22 protein expression and/or activity, said RNAi targeting exon 5 of a nucleic acid sequence encoding said PMP22 protein, said method comprising at least the steps of:
a) contacting an RNAi molecule candidate with a host cell expressing a PMP22 protein under conditions liable to allow an inhibition of expression of said protein,
b) measuring a level of expression of said protein PMP22,
c) comparing the level of expression obtained at step b) with a reference level of expression obtained with at least one of RNAi molecules comprising an antisense nucleic acid sequence selected in the group consisting of SEQ ID NO:36; SEQ ID NO: 54; SEQ ID NO: 55; SEQ ID NO: 59; SEQ ID NO: 60; SEQ ID NO: 61 and SEQ ID NO: 65, and
d) selecting an RNAi molecule candidate able to inhibit the expression of protein PMP22 to a level being substantially the same than the level of expression obtained with at least one of RNAi molecules comprising an antisense nucleic acid sequence selected in the group consisting of SEQ ID NO:36; SEQ ID NO: 54; SEQ ID NO: 55; SEQ ID NO: 59; SEQ ID NO: 60; SEQ ID NO: 61 and SEQ ID NO: 65.

The measure of the expression level of the PMP22 gene may be made, for example, by measuring the expression level of the mRNA or the protein. Measure of expression level of the mRNA or of the protein may be made by any known techniques in the art.

The measure of expression level of the mRNA may be quantification of a band on a Northern blot or by RT-qPCR.

The measure of the expression level of the protein may be made, for example, by Western blot or by immunohistochemistry.

The invention will be further illustrated by the following figures and examples. However, these examples and figures should not be interpreted in any way as limiting the scope of the present invention.

The following items are also herein disclosed:

Item 1: An RNA interferent (RNAi) molecule that inhibits a PMP22 protein expression and/or activity, said RNAi targeting exon 5 of a nucleic acids sequence encoding said PMP22 protein.

Item 2: The RNAi according to item 1, wherein said RNAi targets a region comprising or consisting in the nucleic acids sequence ranging from position 638 to position 690 of SEQ ID NO: 79.

Item 3: The RNAi molecule according to item 1 or 2, wherein said RNAi is selected in the group consisting of siRNA, miRNA, dsRNA, and shRNA.

Item 4: The RNAi molecule according to anyone of items 1 to 3, wherein said RNAi has:
a ΔH ranging from about −148.2 to about −138.4 kcal/mol,
a ΔS ranging from about −0.39 to about −0.37 kcal/° K/mol,
a Tm ranging from about 49.58 to about 56.34° C.,
a GC content ranging from about 42.86 to about 52.38%,
a ΔG ranging from about −32.88 to about −29.19 kcal/mol,
a 3′-end stability ranging from about −11.16 to about −6.58 kcal/mol, and/or
a 5′-end ΔG ranging from about −11.4 to about −5.73 kcal/mol.

Item 5: The RNAi molecule according to anyone of items 1 to 4, wherein said RNAi comprises an antisense nucleic acids sequence selected in the group consisting of SEQ ID NO: 55; SEQ ID NO: 56; SEQ ID NO: 60; SEQ ID NO: 61; SEQ ID NO: 62 and SEQ ID NO: 66, in particular in the group consisting in SEQ ID NO: 60 and SEQ ID NO: 61; and in particular said RNAi comprises a nucleic acids sequence of SEQ ID NO: 61.

Item 6: A nucleic acids delivery system comprising a nucleic acids sequence consisting in or coding for an RNAi molecule according to anyone of items 1 to 5.

Item 7: The nucleic acids delivery system according to item 6, wherein said delivery system comprises an expression vector selected in a group consisting of a viral or a non-viral expression vector.

Item 8: The nucleic acids delivery system according to item 7, wherein said expression vector is a viral expression vector selected in the group consisting in adeno-associated virus, adenovirus, retrovirus, herpes simplex virus, vaccinia virus, SV40-type virus, polyoma virus, Epstein-Barr virus, papilloma virus, lentivirus, and poliovirus.

Item 9: The nucleic acids delivery system according to any one of items 6 to 8, wherein said delivery system is a viral expression vector selected in the group consisting in AAV9, AAV2/9, AAV10, AAVrh10 and AAV2/rh10.

Item 10: An isolated host cell containing an RNAi molecule according to anyone of items 1 to 5, or a nucleic acids delivery system according to anyone of items 6 to 9.

Item 11: A pharmaceutical composition comprising an RNAi molecule according to anyone of items 1 to 5, or a nucleic acids delivery system according to anyone of items 6 to 9, and a pharmaceutically acceptable excipient.

Item 12: An RNAi molecule according to anyone of items 1 to 5, or a nucleic acids delivery system according to anyone of items 6 to 9, as a medicament.

Item 13: An RNAi molecule according to anyone of items 1 to 5, or a nucleic acids delivery system according to anyone of items 6 to 9, or a pharmaceutical composition according to item 11, for use in preventing and/or treating a Charcot-Marie-Tooth type 1A or a Charcot-Marie-Tooth type 1E disease in a patient in need thereof.

Item 14: An RNAi molecule, a nucleic acids delivery system, or a pharmaceutical composition for use according to item 13, wherein said RNAi molecule, said delivery system, or said pharmaceutical composition is administered by systemic route.

Item 15: An adeno-associated virus (AAV) vector comprising an RNAi molecule comprising an antisense nucleic acids sequence of SEQ ID NO: 61.

It is also disclosed a method for screening an RNAi molecule for preventing or treating a CMT1A or 1E diseases, the method comprising: (a) contacting an RNAi molecule candidate to cells comprising a gene encoding the PMP22 protein; and (b) measuring the expression level of the gene encoding the PMP22 protein, when the candidate decrease the expression level of the gene of PMP22 protein, it is determined as an RNAi molecule for preventing or treating the CMT1A or 1E diseases.

It is further disclosed a method of selecting an RNA interferent (RNAi) molecule that inhibits a PMP22 protein expression and/or activity, said RNAi targeting exon 5 of a nucleic acid sequence encoding said PMP22 protein, said method comprising at least the steps of:
a) contacting an RNAi molecule candidate with a host cell expressing a PMP22 protein under conditions liable to allow an inhibition of expression of said protein,
b) measuring a level of expression of said protein PMP22,
c) comparing the level of expression obtained at step b) with a reference level of expression obtained with at least one of RNAi molecules comprising an antisense nucleic acid sequence selected in the group consisting of SEQ ID NO:36; SEQ ID NO: 55; SEQ ID NO: 56; SEQ ID NO: 60; SEQ ID NO: 61; SEQ ID NO: 62 and SEQ ID NO: 66, and
d) selecting an RNAi molecule candidate able to inhibit the expression of protein PMP22 to a level being substantially the same than the level of expression obtained with at least one of RNAi molecules comprising an antisense nucleic acid sequence selected in the group consisting of SEQ ID NO:36; SEQ ID NO: 55; SEQ ID NO: 56; SEQ ID NO: 60; SEQ ID NO: 61; SEQ ID NO: 62 and SEQ ID NO: 66.

TABLE OF
SEQUENCES

| SEQ | | |
|---|---|---|
| ID | | |
| NO | Sequence function | Sequence |
| 1 | sense sequence of RNAi #1 targeting hPMP22 | CCCUGGCCCUUCUCAGCGGUG |
| 2 | sense sequence of RNAi #2 targeting hPMP22 | CCUGGCCCUUCUCAGCGGUGU |
| 3 | sense sequence of RNAi #3 targeting hPMP22 | CUGGCCCUUCUCAGCGGUGUC |
| 4 | sense sequence of RNAi #4 targeting hPMP22 | UGGCCCUUCUCAGCGGUGUCA |
| 5 | sense sequence of RNAi #5 targeting hPMP22 | GGCCCUUCUCAGCGGUGUCAU |
| 6 | sense sequence of RNAi #6 targeting hPMP22 | GCCCUUCUCAGCGGUGUCAUC |
| 7 | sense sequence of RNAi #7 targeting hPMP22 | CCCUUCUCAGCGGUGUCAUCU |
| 8 | sense sequence of RNAi #8 targeting hPMP22 | CCUUCUCAGCGGUGUCAUCUA |
| 9 | sense sequence of RNAi #9 targeting hPMP22 | CUUCUCAGCGGUGUCAUCUAU |
| 10 | sense sequence of RNAi #10 targeting hPMP22 | UUCUCAGCGGUGUCAUCUAUG |
| 11 | sense sequence of RNAi #11 targeting hPMP22 | UCUCAGCGGUGUCAUCUAUGU |
| 12 | sense sequence of RNAi #12 targeting hPMP22 | CUCAGCGGUGUCAUCUAUGUG |
| 13 | sense sequence of RNAi #13 targeting hPMP22 | UCAGCGGUGUCAUCUAUGUGA |
| 14 | sense sequence of RNAi #14 targeting hPMP22 | CAGCGGUGUCAUCUAUGUGAU |
| 15 | sense sequence of RNAi #15 targeting hPMP22 | AGCGGUGUCAUCUAUGUGAUC |
| 16 | sense sequence of RNAi #16 targeting hPMP22 | GCGGUGUCAUCUAUGUGAUCU |
| 17 | sense sequence of RNAi #17 targeting hPMP22 | CGGUGUCAUCUAUGUGAUCUU |
| 18 | sense sequence of RNAi #18 targeting hPMP22 | GGUGUCAUCUAUGUGAUCUUG |
| 19 | sense sequence of RNAi #19 targeting hPMP22 | GUGUCAUCUAUGUGAUCUUGC |
| 20 | sense sequence of RNAi #20 targeting hPMP22 | UGUCAUCUAUGUGAUCUUGCG |
| 21 | sense sequence of RNAi #21 targeting hPMP22 | GUCAUCUAUGUGAUCUUGCGG |
| 22 | sense sequence of RNAi #22 targeting hPMP22 | UCAUCUAUGUGAUCUUGCGGA |
| 23 | sense sequence of RNAi #23 targeting hPMP22 | CAUCUAUGUGAUCUUGCGGAA |
| 24 | sense sequence of RNAi #24 targeting hPMP22 | AUCUAUGUGAUCUUGCGGAAA |
| 25 | sense sequence of RNAi #25 targeting hPMP22 | UCUAUGUGAUCUUGCGGAAAC |
| 26 | sense sequence of RNAi #26 targeting hPMP22 | CUAUGUGAUCUUGCGGAAACG |
| 27 | sense sequence of RNAi #27 targeting hPMP22 | UAUGUGAUCUUGCGGAAACGC |
| 28 | sense sequence of RNAi #28 targeting hPMP22 | AUGUGAUCUUGCGGAAACGCG |
| 29 | sense sequence of RNAi #29 targeting hPMP22 | UGUGAUCUUGCGGAAACGCGA |
| 30 | sense sequence of RNAi #30 targeting hPMP22 | GUGAUCUUGCGGAAACGCGAA |
| 31 | sense sequence of RNAi #31 targeting hPMP22 | UGAUCUUGCGGAAACGCGAAU |
| 32 | sense sequence of RNAi #32 targeting hPMP22 | GAUCUUGCGGAAACGCGAAUG |
| 33 | sense sequence of RNAi #33 targeting hPMP22 | AUCUUGCGGAAACGCGAAUGA |
| 34 | sense sequence of shRNA1 targeting hPMP22 | CCTGTTCTTCTGCCAACTCTT |
| 35 | sense sequence of shRNA2 targeting hPMP22 | GGCAATGGACACGCAACTGAT |
| 36 | sense sequence of shRNA3 targeting hPMP22 | CGGTGTCATCTATGTGATCTT |
| 37 | sense sequence of shRNA4 targeting hPMP22 | TGTCGATCATCTTCAGCATTC |
| 38 | sense sequence of shRNA5 targeting hPMP22 | CACGATCGTCAGCCAATGGAT |
| 39 | sense sequence of RNAi #11B targeting cPMP22 | UCUCAGCGGUGUCAUCUACGU |
| | (Macaca fascicularis) | |
| 40 | sense sequence of RNAi #12B targeting cPMP22 | CUCAGCGGUGUCAUCUACGUG |
| | (Macaca fascicularis) | |
| 41 | sense sequence of RNAi #16B targeting cPMP22 | GCGGUGUCAUCUACGUGAUCU |
| | (Macaca fascicularis) | |
| 42 | sense sequence of RNAi #17B targeting cPMP22 | CGGUGUCAUCUACGUGAUCUU |
| | (Macaca fascicularis) | |
| 43 | sense sequence of RNAi #18B targeting cPMP22 | GGUGUCAUCUACGUGAUCUUG |
| | (Macaca fascicularis) | |
| 44 | sense sequence of RNAi #22B targeting cPMP22 | UCAUCUACGUGAUCUUGCGGA |
| | (Macaca fascicularis) | |
| 45 | antisense sequence of RNAi #1 targeting hPMP22 | CACCGCUGAGAAGGGCCAGGG |
| 46 | antisense sequence of RNAi #2 targeting hPMP22 | ACACCGCUGAGAAGGGCCAGG |
| 47 | antisense sequence of RNAi #3 targeting hPMP22 | GACACCGCUGAGAAGGGCCAG |
| 48 | antisense sequence of RNAi #4 targeting hPMP22 | UGACACCGCUGAGAAGGGCCA |
| 49 | antisense sequence of RNAi #5 targeting hPMP22 | AUGACACCGCUGAGAAGGGCC |
| 50 | antisense sequence of RNAi #6 targeting hPMP22 | GAUGACACCGCUGAGAAGGGC |
| 51 | antisense sequence of RNAi #7 targeting hPMP22 | AGAUGACACCGCUGAGAAGGG |
| 52 | antisense sequence of RNAi #8 targeting hPMP22 | UAGAUGACACCGCUGAGAAGG |
| 53 | antisense sequence of RNAi #9 targeting hPMP22 | AUAGAUGACACCGCUGAGAAG |
| 54 | antisense sequence of RNAi #10 targeting hPMP22 | CAUAGAUGACACCGCUGAGAA |
| 55 | antisense sequence of RNAi #11 targeting hPMP22 | ACAUAGAUGACACCGCUGAGA |
| 56 | antisense sequence of RNAi #12 targeting hPMP22 | CACAUAGAUGACACCGCUGAG |
| 57 | antisense sequence of RNAi #13 targeting hPMP22 | UCACAUAGAUGACACCGCUGA |
| 58 | antisense sequence of RNAi #14 targeting hPMP22 | AUCACAUAGAUGACACCGCUG |
| 59 | antisense sequence of RNAi #15 targeting hPMP22 | GAUCACAUAGAUGACACCGCU |
| 60 | antisense sequence of RNAi #16 targeting hPMP22 | AGAUCACAUAGAUGACACCGC |
| 61 | antisense sequence of RNAi #17 targeting hPMP22 | AAGAUCACAUAGAUGACACCG |
| 62 | antisense sequence of RNAi #18 targeting hPMP22 | CAAGAUCACAUAGAUGACACC |
| 63 | antisense sequence of RNAi #19 targeting hPMP22 | GCAAGAUCACAUAGAUGACAC |
| 64 | antisense sequence of RNAi #20 targeting hPMP22 | CGCAAGAUCACAUAGAUGACA |
| 65 | antisense sequence of RNAi #21 targeting hPMP22 | CCGCAAGAUCACAUAGAUGAC |
| 66 | antisense sequence of RNAi #22 targeting hPMP22 | UCCGCAAGAUCACAUAGAUGA |
| 67 | antisense sequence of RNAi #23 targeting hPMP22 | UUCCGCAAGAUCACAUAGAUG |
| 68 | antisense sequence of RNAi #24 targeting hPMP22 | UUUCCGCAAGAUCACAUAGAU |
| 69 | antisense sequence of RNAi #25 targeting hPMP22 | GUUUCCGCAAGAUCACAUAGA |
| 70 | antisense sequence of RNAi #26 targeting hPMP22 | CGUUUCCGCAAGAUCACAUAG |
| 71 | antisense sequence of RNAi #27 targeting hPMP22 | GCGUUUCCGCAAGAUCACAUA |
| 72 | antisense sequence of RNAi #28 targeting hPMP22 | CGCGUUUCCGCAAGAUCACAU |
| 73 | antisense sequence of RNAi #29 targeting hPMP22 | UCGCGUUUCCGCAAGAUCACA |
| 74 | antisense sequence of RNAi #30 targeting hPMP22 | UUCGCGUUUCCGCAAGAUCAC |
| 75 | antisense sequence of RNAi #31 targeting hPMP22 | AUUCGCGUUUCCGCAAGAUCA |
| 76 | antisense sequence of RNAi #32 targeting hPMP22 | CAUUCGCGUUUCCGCAAGAUC |
| 77 | antisense sequence of RNAi #33 targeting hPMP22 | UCAUUCGCGUUUCCGCAAGAU |
| 78 | scrambled (control) RNA sequence | AGTTCCAGTACGGCTCCAA |
| 79 | mRNA sequence of hPMP22 (NCBI Reference | agttacagggagcaccaccag |
| | Sequence: NM_000304.4) | ggaacatctcggggagcctgg |
| | | ttggaagctgcaggcttagtc |
| | | tgtcggctgcgggtctctgac |
| | | tgccctgtggggagggtcttg |
| | | ccttaacatcccttgcatttg |
| | | gctgcaaagaaatctgcttgg |
| | | aagaaggggttacgctgtttg |
| | | gccgggcagaaactccgctga |
| | | gcagaacttgccgccagaatg |
| | | ctcctcctgttgctgagtatc |
| | | atcgtcctccacgtcgcggtg |
| | | ctggtgctgctgttcgtctcc |
| | | acgatcgtcagccaatggatc |
| | | gtgggcaatggacacgcaact |
| | | gatctctggcagaactgtagc |
| | | acctcttcctcaggaaatgtc |
| | | caccactgtttctcatcatca |
| | | ccaaacgaatggctgcagtct |
| | | gtccaggccaccatgatcctg |
| | | tcgatcatcttcagcattctg |
| | | tctctgttcctgttcttctgc |
| | | caactcttcaccctcaccaag |
| | | gggggcaggttttacatcact |
| | | ggaatcttccaaattcttgct |
| | | ggtctgtgcgtgatgagtgct |
| | | gcggccatctacacggtgagg |
| | | cacccggagtggcatctcaac |
| | | teggattactcctacggtttc |
| | | gcctacatcctggcctgggtg |
| | | gccttccccctggcccttctc |
| | | agcggtgtcatctatgtgatc |
| | | ttgcggaaacgcgaatgaggc |
| | | gcccagacggtctgtctgagg |
| | | ctctgagcgtacatagggaag |
| | | ggaggaagggaaaacagaaag |
| | | cagacaaagaaaaaagagcta |
| | | gcccaaaatcccaaactcaaa |
| | | ccaaaccaaacagaaagcagt |
| | | ggaggtgggggttgctgttga |
| | | ttgaagatgtatataatatct |
| | | ccggtttataaaacctattta |
| | | taacactttttacatatatgt |
| | | acatagtattgtttgcttttt |
| | | atgttgaccatcagcctcgtg |
| | | ttgagccttaaagaagtagct |
| | | aaggaactttacatcctaaca |
| | | gtataatccagctcagtattt |
| | | ttgttttgttttttgtttgtt |
| | | tgttttgttttacccagaaat |
| | | aagataactccatctcgcccc |
| | | ttccctttcatctgaaagaag |
| | | atacctccctcccagtccacc |
| | | tcatttagaaaaccaaagtgt |
| | | gggtagaaaccccaaatgtcc |
| | | aaaagcccttttctggtgggt |
| | | gacccagtgcatccaacagaa |
| | | acagccgctgcccgaacctct |
| | | gtgtgaagctttacgcgcaca |
| | | cggacaaaatgcccaaactgg |
| | | agcccttgcaaaaacacggct |
| | | tgtggcattggcatacttgcc |
| | | cttacaggtggagtatcttcg |
| | | tcacacatctaaatgagaaat |
| | | cagtgacaacaagtctttgaa |
| | | atggtgctatggatttaccat |
| | | tccttattatcactaatcatc |
| | | taaacaactcactggaaatcc |
| | | aattaacaattttacaacata |
| | | agatagaatggagacctgaat |
| | | aattctgtgtaatataaatgg |
| | | tttataactgcttttgtacct |
| | | agctaggctgctattattact |
| | | ataatgagtaaatcataaagc |
| | | cttcatcactcccacattttt |
| | | cttacggtcggagcatcagaa |
| | | caagcgtctagactccttggg |
| | | accgtgagttcctagagcttg |
| | | gctgggtctaggctgttctgt |
| | | gcctccaaggactgtctggca |
| | | atgacttgtattggccaccaa |
| | | ctgtagatgtatatatggtgc |
| | | ccttctgatgctaagactcca |
| | | gaccttttgtttttgctttgc |
| | | attttctgattttataccaac |
| | | tgtgtggactaagatgcatta |
| | | aaataaacatcagagtaactc |
| | | a |
| 80 | Exon 5 of mRNA sequence of hPMP22 (NCBI | CCCTGGCCCTTCTCAGCGGTGTC |
| | Reference Sequence: NM_000304.4) | ATCTATGTGATCTTGCGGAAACG |
| | | CGAATGA |
| 81 | Examplary primer sequence | ATCGATACGTAG |

### EXAMPLES

The present invention is further illustrated by the following examples.

### Example 1: Materials and Methods

### Cloning and Vector Production

RNAi molecules production was carried out through solid-phase synthesis (Dong Y, Siegwart D J, Anderson D G. Strategies, design, and chemistry in siRNA delivery systems. Adv Drug Deliv Rev. 2019; 144: 133-147). RNA synthesis is a repetitive chemical cycle in which each nucleotide is added on a solid support. This cycle starts with a deprotection step to remove the protective group on 5′-hydroxyl of the solid support bound nucleotide. The resulting 5′-hydroxyl is then coupled with an activated 3′-phosphorous ester, followed by a capping step to remove the unreacted nucleotides from the reaction system. The intermediate undergoes another step to oxidize phosphite to phosphorous ester. After the chain assembly, the oligomer is released from the solid support, deprotected, and purified by HPLC. Two types of building blocks are used including 2′-O-TOM and 2′-O-ACE modified nucleotides. Both methods provide a coupling yield of over 99%. The whole process is automated by utilizing oligonucleotide synthesizers.

shRNA targeting human PMP22 mRNA and control (scrambled) shRNA were cloned under the control of U6 promoter in a pAAV plasmid using synthetic oligonucleotides that contain sense and antisense sequences linked with a loop (McIntyre, G. J., Fanning, G. C. Design and cloning strategies for constructing shRNA expression vectors. BMC Biotechnol 6, 1 (2006)). When these oligonucleotides are hybridized the DNA duplex bears cohesive extremities for EcoR1 and Bgl2 restriction sites which allows their cloning into pAAV plasmid opened with the EcoR1 and Bgl2 enzymes.

The pAAV plasmids were further used to generate AAV2/9-RNAi vectors.

Vector production was performed following the CPV facility protocol (Ayuso, E., Mingozzi, F. & Bosch, F. Production, purification and characterization of adeno-associated vectors. CGT 10, 423-436 (2010)). Briefly, recombinant AAVs were manufactured by co-transfection of HEK293 cells and purified by cesium chloride density gradients followed by extensive dialysis against phosphate-buffered saline (PBS). Vector titers were determined by qPCR, the target amplicons correspond to the inverted terminal repeat (ITR) sequences, ITR-2.

All RNAi sequences used in the different assays are presented in TABLE OF SEQUENCES.

shRNAs 1 and 2 were disclosed under the name shRNA A and B respectively in Gautier, B., Hajjar, H., Soares, S. et al. AAV2/9-mediated silencing of PMP22 prevents the development of pathological features in a rat model of Charcot-Marie-Tooth disease 1 A. Nat Commun 12, 2356 (2021)).

### Cell Culture

One day before transfection, 3·10⁵ HEK293T cells were seeded in a 6-well-plate in the appropriate amount of growth medium DMEM high glucose 10% FBS without antibiotics such that they were 80-90% confluent at the time of transfection. Cells were cultured at 37° C. in a CO₂ incubator before transfection.

### Lipofectamin™ Transfection and Viral Infection

### Screening of the RNAi Molecules

The screening of RNAi molecules to silence human PMP22 expression was carried by co-transfecting HEK293 cells with a human-flag vector pCMV3-hPMP22-Flag (HG14519-CF, Sinobiological—cDNA molecule) or with a cynomolgus-flag vector pCMV3-cPMP22-Flag (SB Sino Biological CG90941-CF G13SE06M013—cDNA molecule), and with RNAi molecules (see TABLE OF SEQUENCES) in pAAV vectors using Lipofectamin 2000 reagent and following INVITROGEN procedure: Protocol Pub. No. MAN0007824 Rev.1.0 (https://assets.thermofisher.com/TFS-Assets/LSG/manuals/Lipofectamine_2000_Reag_protocol.pdf).

For each transfection sample, cDNA-RNAi molecule-Lipofectamine™ 2000 complexes were prepared as follows:

a. cDNA (750 ng in sterile water) and RNAi molecules (20 μM stock in 1×RNA annealing/Dilution buffer (ThermoFisher Cat. no. 13778-075) were diluted in Opti-MEM® I Medium (Thermo Fisher Scientific, Catalog nos. 11668-027 or 11668-019) without serum according to the following parameters:

TABLE 1

| Volume of | | | Opti-MEM | Lipofectamin | DMEM |
|---|---|---|---|---|---|
| plating | Plasmid | RNAi | dilution | 2000 | HG |
| medium | DNA | vector | volume | volume | 10% FBS |
| 2 mL | 750 ng | 3 μg | 250 μL | 5 μL | 1745 μL |

b. Lipofectamine™ 2000 (Lipid) was mixed before use, and then diluted with the appropriate amount in Opti-MEM® I Medium without serum. The overall was mixed and incubated for 5 minutes at room temperature.

c. The diluted cDNA and RNAi molecule were combined with the diluted Lipofectamine™ 2000 and incubated for 20 minutes at room temperature to allow complex formation to occur.

The cDNA-RNAi molecule Lipofectamine™ 2000 complexes were added to each well containing cells and medium. The plates were incubated at 37° C. in a C02 incubator during 48h, and then the cells were harvested. Samples were frozen at −20° C. before Western blotting. Negative control, cells transfected with shRNA scramble and a positive control (cells transfected with shRNA3) were used during each transfection series. The wild-type (WT) HEK293 cells (without siRNA transfection) were also used as control.

Total protein concentration was quantified using bicinchoninic acid method (Pierce REF 23225 Lot Num SK258363), adjusted at 1 μg of total protein/μl and then frozen at −20° C. before flag-PMP22 and β-tubulin Western Blot quantification.

Experiments were carried out in triplicate.

Testing with an AAV9-Vector

Validation of the efficiency of selected RNAi with AAV9-vectors expressing RNAi molecules to silence human or cynomolgus PMP22 expression was done by transfecting HEK293 cells with a human-flag vector pCMV3-hPMP22-Flag (HG14519-CF, Sinobiological—cDNA molecule) or a cynomolgus-flag vector pCMV3-cPMP22-Flag (SB Sino Biological CG90941-CF G13SE06M013—cDNA molecule), and then infecting the cells with an AAV9-vector expressing an RNAi molecule. A shRNA scramble was used as control. Depending on the experiments, three (on cPMP22) or four (on hPMP22) doses of viruses were tested: 2·10⁸ vg/well; 10⁸ vg/well; 5·10⁷ vg/well; and 10⁷ vg/well.

The formulations for the transfection of hPMP22-Flag or cPMP22-Flag in 6-wells plates were as follows:

TABLE 2

| Volume of | | Opti-MEM | Lipofectamin | DMEM |
|---|---|---|---|---|
| plating | Plasmid | dilution | 2000 | HG 10% |
| medium | DNA | volume | volume | FBS |
| 2 mL | 750 ng | 250 μL | 5 μL | 1745 μL |

Before transfection with hPMP22-Flag or cPMP22-Flag, 3·10⁵ HEK293 cells were seeded in 6-wells plates in the appropriate amount of DMEM high glucose 10% FBS without antibiotics so that they were 80-90% confluent at the time of transfection. One day after seeding, the cells were transfected with the corresponding plasmid. 24 hours after transfection, the transfected HEK293 cells were infected with the corresponding doses of AAV9-vector expressing an RNAi molecule.

For each transfection sample, cDNA molecule-Lipofectamine™ 2000 complexes were prepared as follows:

a. cDNA diluted in Opti-MEM® I Medium without serum (concentrations indicated in the table above).

b. Lipofectamine™ 2000 was gently mixed before use, then diluted in the appropriate amount in Opti-MEM® I Medium without serum, mixed gently and incubated for 5 minutes at room temperature.

c. The diluted DNA was combined with the diluted Lipofectamine™ 2000, mixed gently and incubated for 20 minutes at room temperature to allow complex formation to occur.

The cDNA-Lipofectamine™ 2000 complexes were added to each well containing cells and medium, and then mixed gently by rocking the plate back and forth. The cells were incubated at 37° C. in a CO₂ incubator during 24h.

24 hours after cell transfection, the AAV9 particles were diluted in PBS and added at the corresponding viral titer. The cells were incubated at 37° C. in a CO₂ incubator during 48h.

The transfected cells were harvested 48 hours after viral infection, and then lysed. Total protein concentration was quantified using bicinchoninic acid method (Pierce), adjusted at 1 μg of total protein/μl and then frozen at −20° C. before flag-PMP22 and β-tubulin Western Blot quantification.

Each experiment was carried out in triplicate.

### PMP22 Western Blot Analysis

Harvested cells were sampled and rinsed and then solubilized in RIPA lysis buffer completed with protease inhibitors (Fisher Scientific, France) and homogenized on a rotating wheel at 4° C. for 3 h. Cells were then sonicated three times during 10 s on ice (Microson ultrasonic cell disruptorXL, Microsonic) and centrifuged for 30 min at 10 000 rpm at 4° C. Proteins concentrations from cell lysates were quantified using the Bicinchoninic acid (BCA) protein assay kit (Thermo Scientific, France).

Ten micrograms of proteins were loaded on a 4-20% precast SDS-polyacrylamide gel (MiniProtean gels, Bio Rad, France). Proteins were transferred to pre-blocked nitrocellulose membranes (Bio Rad Trans blot transfer pack) through semi-dry transfer process (BioRad Bio Rad Trans-Blot Turbo system). Membranes were first incubated with the REVERT total protein stain solution (LI-COR Biosciences, France) to record the overall amount of protein per lane (Pillai-Kastoori, L., Schutz-Geschwender, A. R. & Harford, J. A. A systematic approach to quantitative Western blot analysis. Anal. Biochem. 593, 113608 (2020)). Then, membranes were blocked for 1 h at room temperature using LI-COR blocking buffer (Odyssey Blocking buffer, LI-COR Biosciences, France).

Membranes were incubated with the following primary antibodies overnight at 4° C. in the same blocking buffer: mouse anti-flag (1:1000, Sigma-Aldrich, F1804) and rabbit anti-β-tubulin (1:1000, Sigma Aldrich, ZRB1416).

Following three washes of 10 min with TBS-0.1% V/V Tween-20 (TBST), and the incubated for 1 hour at room temperature with the secondary fluorescence antibodies: donkey anti-rabbit IRDye 800 (1:10.000, LI-COR Biosciences, 925-32213) and donkey anti-mouse IRDye 680 (1:10.000, LI-COR Biosciences, 925-68072). After three washes in TBST, visualization of the bands was carried out with the Odyssey CLX LI-COR Imaging System and its “Image Studio” software. Band quantification was performed using Image J software (version 4.0).

Results are means of experiments carried out in triplicate. Depending on the experiments, and where indicated, results were expressed by being normalized over the expression of PMP22 obtained in presence of the control (scrambled) RNAi molecule or over the expression of b-tubulin.

siRNA Characterization

All of the formulas used in the program are given below for quick reference:

Melting Temperature: The melting temperature is calculated using the formula based on the nearest neighbor thermodynamic theory. It is the temperature at which half of the oligonucleotides are bonded. The formula is from the paper by Freier et al. (1986) Proc. Natl. Acad. Sci. U.S.A. 83, 9373-9377. These are the latest and most accurate nearest neighbor-based Tm calculations. Tm=ΔH/(ΔS+R*ln(C/4))+16.6 log ([K+]/(1+0.7 [K+]))−273.15. ΔH: is enthalpy for helix formation. ΔS is entropy for helix formation. R is molar gas constant (1.987 cal/° C.*mol) C is the nucleic acids concentration. [K+] is salt concentration. Example: Say the primer sequence is ATCGATACGTAG (SEQ ID NO: 81). The ΔH and ΔS values of this primer will be 6-85000 cal/mol and −234.7 cal/° K/mol respectively (as calculated below). After substituting all the values, the Tm value of this primer will be 16.69° K.

GC %: GC % is the percentage of G and C in the primer. It is calculated by dividing the sum of G and C with the total number of bases present in the primer. Example: Say the primer sequence is ATCGATACGTAG (SEQ ID NO: 81). The GC % of this primer will be (5/12*100)=41.67%.

ΔG: This is the free energy of the primer calculated using the nearest neighbor method of Breslauer et al. (1986) Proc. Natl. Acad. Sci. U.S.A. 83, 3746-3750. ΔG is calculated by the formula ΔG=ΔH−TΔS. Here ΔH is the enthalpy of primer, T is the temperature, ΔS is the entropy of primer. T is set by ΔG temp. in the preferences. First the ΔH and ΔS are calculated and then the ΔG is calculated using their values. Example: Say the primer sequence is ATCGATACGTAG (SEQ ID NO: 81). Its ΔH and ΔS will be −85000 cal/mol and 7-234.7 cal/° K/mol respectively (as calculated below). Its ΔG will be −85000−(298.15*−234.7)=−15024.195 cal/mol=−15.02 kcal/mol.

3′ end stability: The stability of the primer determines its false priming efficiency. An ideal primer has a stable 5′ end and an unstable 3′ end. If the primer has a stable 3′ end, it will bond to a site which is complementary to it other than the target with its 5′ end hanging off the edge. It may then lead to secondary bands. Primers with low stability at the 3′ ends function well because the 3′ end bonding to false priming sites are too unstable to extend. The 3′ end stability is the ΔG value of the 5 bases of primer taken from 3′ end. The lower this value, numerically, the more liable the primer is to show secondary bands. Example: Say the primer sequence is ATCGATACGTAG (SEQ ID NO: 81). Its 3′ end stability will be ΔG(CGTAG)=ΔH(CGTAG)−298.15*ΔS(CGTAG). The ΔH and ΔS will be −32200 cal/mol and −82.8 cal/° K/mol resp. Thus its 3′ end stability will be −32200−(298.15*−82.8)=−7513.18 cal/mol=−7.51 kcal/mol.

ΔH: This is the enthalpy of the primer as calculated by the nearest neighbor method of Breslauer et al. (1986) Proc. Natl. Acad. Sci. U.S.A. 83, 3746-3750. ΔH for a pentamer is calculated as follows: ΔHATGCA=ΔHAT+ΔHTG+ΔHGC+ΔHCA. Example: Say the primer sequence is ATCGATACGTAG (SEQ ID NO: 81). Its ΔH will be (8600+5600+11900+5600+8600+6000+6500+11900+6500+6000+7800)=−85000 cal/mol=−85 kcal/mol.

ΔS: This is the entropy of the primer as calculated by the nearest neighbor method of Breslauer et al. (1986) Proc. Natl. Acad. Sci. U.S.A. 83, 3746-3750. ΔS for a pentamer is calculated as follows: ΔSATGCA=ΔSAT+ΔSTG+ΔSGC+ΔSCA. An initiation value of 15.1 is added to the ΔS calculation. Example: Say the primer sequence is ATCGATACGTAG (SEQ ID NO: 81). Its ΔS will be (23.9+13.5+27.8+13.5+23.9+16.9+17.3+27.8+17.3+16.9+20.8)+15.1=−234.7 cal/° K/mol=−0.23 kcal/° K/mol.

5′-end ΔG: Stability of the 5′ termini allows for efficient bonding of the primer to the target site. This stable 5′ region is called the GC Clamp. It ensures adequate binding of the primer to the template. Use of primers with optimal stability allows for the use of lower annealing temperatures without the production of secondary bands. Notice that the 3′ end should not be very stable and the 5′ end should have a strong GC clamp. The GC Clamp is the ΔG value of the 5 bases of primer taken from 5′ end. The lower this value, numerically, the more efficient is the primer. Example: Say the primer sequence is ATCGATACGTAG (SEQ ID NO: 81). Its 5′ ΔG will be ΔG(ATCGA)=ΔH(ATCGA)−298.15*ΔS(ATCGA).

### Statistical Analysis

For the screening experiments, descriptive statistics by groups were expressed as mean±SEM for continuous variables. Statistical significances were determined using t-student test to compare differences of each siRNA vs positive and negative control. Statistical analyses were performed using GraphPad Prism version 5.02 for Windows, GraphPad Software, La Jolla California USA. A p value of less than 0.05 was considered significant.

For the assays with AAV9-expressing RNAi molecules, descriptive statistics by groups were expressed as mean±SEM for continuous variables. Statistical significances were determined using 2-way ANOVA, followed by a Bonferroni multiple comparisons post hoc test, allowing comparisons between groups versus control at the same concentration, assuming the normal distribution of the variable and the variance homoscedasticity. Statistical analyses were performed using GraphPad Prism version 5.02 for Windows, GraphPad Software, La Jolla California USA. A p value of less than 0.05 was considered significant.

### Example 2: Selection of hPMP22 Target Sequence

The purpose of the study was to screen and select RNAi molecules to develop a gene therapy based on an adeno-associated vector, AAV9, administered directly into the nerve, to treat an inherited disease of the nerve: CMT1A or CMT1E. The therapeutic vector expressing an RNAi molecule will specifically lead to a decrease, or even a suppression, of the expression of the PMP22 protein and will result in preventing or treating the CMT1A or 1E diseases.

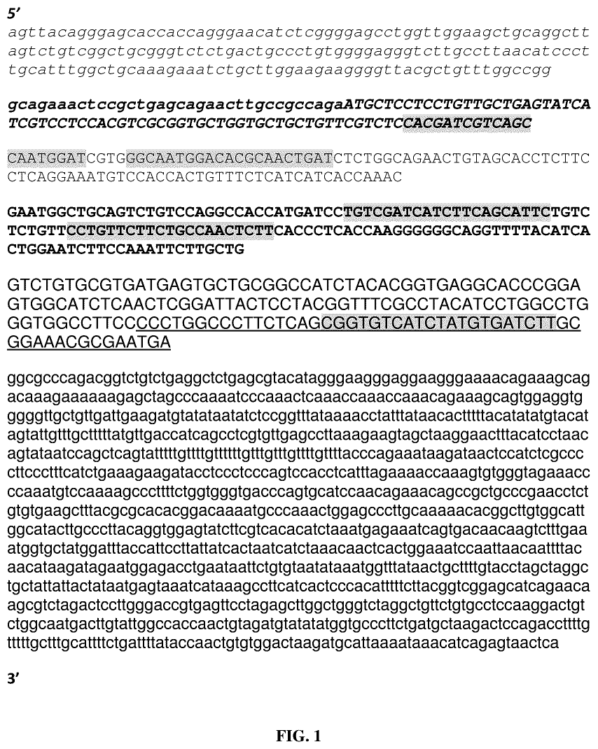

In a first set of experiments, 5 pAAV plasmids expressing inhibitory RNAs (shRNAs: Sh1, Sh2, Sh3, Sh4 and Sh5—see TABLE OF SEQUENCES) directed against different parts of the mRNA coding for the human PMP22 protein (see FIG. 1) were tested as indicated in the Materials & Methods (see “Screening of the RNAi molecules”).

shRNAs Sh1 and Sh2 were disclosed in Gautier, B., Hajjar, H., Soares, S. et al. AAV2/9-mediated silencing of PMP22 prevents the development of pathological features in a rat model of Charcot-Marie-Tooth disease 1 A. Nat Commun 12, 2356 (2021). https://doi.org/10.1038/s41467-021-22593-3.

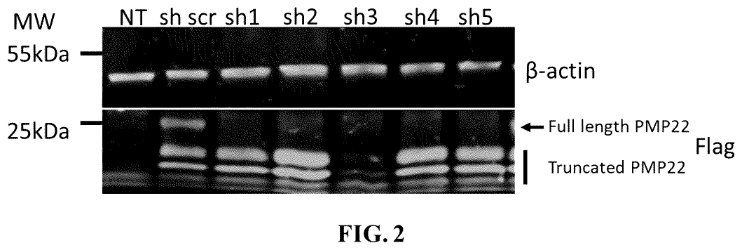

As shown on FIG. 2, all 5 sequences proved to be effective. However, it was surprisingly observed that targeting the region of exon 5 (C-terminal of the PMP22 protein sequence) was the most efficient and the most suitable area to obtain an inhibitory RNA because it allows the decrease in the expression of all forms of the human PMP22 protein (full and truncated forms—see FIG. 2). In this regard, it was observed that the sequences sh1 and sh2 (Gautier et al Nat. Com., 2021) were effective for decreasing the expression of the complete form of human PMP22 (FIG. 2, full PMP22), but were ineffective on the truncated forms. In contrast and surprisingly, shRNA3 targeting exon 5 of PMP22 was the most efficient and was active on truncated forms of PMP22.

Accordingly, targeting exon 5 of the PMP22 protein revealed a particularly interesting strategy for reducing or suppressing the expression of PMP22 for the treatment and prevention of the CMT1A or 1E diseases.

Furthermore, shRNA3 showed to be good candidate to start developing RNAi molecules for therapeutic use in the CMT1A or 1E diseases.

### Example 3: Screening of RNAi Molecules on hPMP22

Further to the identification of exon 5 of PMP22 as a relevant target sequence for reducing and even suppressing the expression of PMP22 for the prevention of treatment of the CMT1A or 1E diseases, and the identification of shRNA3 as a starting RNAi candidate, a series of 33 inhibitory RNA sequences (RNAi #1433; SEQ ID NO:1-33 and SEQ ID NO: 45-77; TABLE OF SEQUENCES) of 21 bp covering the selected area were produced. These RNAi molecules (siRNA) were tested for their effectiveness in reducing or suppressing the expression of human PMP22 in HEK293 cells. shRNA scrambled (SEQ ID NO: 78) and shRNA3 (SEQ ID NO:36) were used, respectively, as negative and positive control.

On day 4 after seeding, at 80-90% confluence, the HEK293 cells were co-transfected with a hPMP22-flag plasmid and RNAi molecules. 48 hours after co-transfection, the cells were harvested, lysed, and the proteins extracted for the Western Blot as indicated above. Before use, the samples were frozen at −20° C. The hPMP22 was quantified using Western Blot targeting flag sequences. Results were expressed as being normalized over the expression of hPMP22 in presence of control RNAi (shRNA scramble).

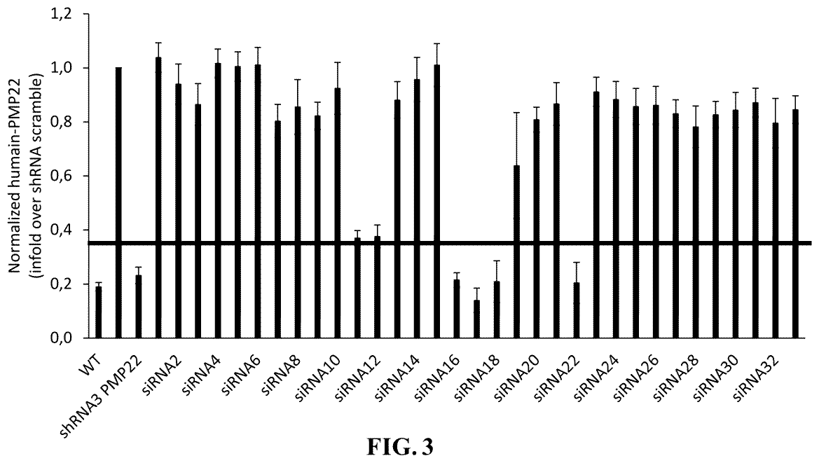

FIG. 3 shows that co-transfection with scrambled shRNA (control) did not affect the expression of flag-human PMP22. The positive control shRNA3 significantly decreased the expression of flag-human PMP22 in co-transfected cells.

Statistically significant decrease of human PMP22-flag expression was observed for the RNAi #11, #12, #16, #17, #18 and #22 after cell co-transfection compared to the negative control. Those RNAi molecules were able to reduce the hPMP22 protein expression by at least 2.5-fold. With these compounds, the flag-human PMP22 expression was reduced down to a level closed to the positive control for these RNAi (shRNA3) or the level of the wild-type (non-transfected) HEK293 cells.

In conclusion, from the 33 tested RNAi molecules, six effective RNAi molecules, i.e., RNAi #11, #12, #16, #17, #18 and #22, were surprisingly identified that efficiently suppress or reduce the expression of hPMP22 in its full-length and N-terminally truncated forms in HEK 293 cells.

### Example 4: Physico-Chemical Characterization of Active RNAi Molecules

The physico-chemical properties of the 33 siRNA were characterized.

The following parameters were measured: Tm, GC %, ΔG in kcal/mol, 3′-end stability Kcal/mol, ΔH in kcal/mol, ΔS in kcal/° K/mol, and 5′-end ΔG in kcal/mol.

The results are summarized in the following TABLE 3:

TABLE 3
physico-chemical characterization of the RNAi molecules

| | | | | 3′-end | | | 5′-end |
|---|---|---|---|---|---|---|---|
| | | | ΔG in | stability | ΔH in | ΔS in | ΔG in |
| RNai | Tm | GC | kcal/ | Kcal/ | kcal/ | kcal/ | kcal/ |
| # | ° C. | % | mol | mol | mol | ° K/mol | mol |
| 1 | 70.59 | 71.43 | −42.5 | −9.69 | −175.5 | −0.45 | −9.98 |
| 2 | 68.14 | 66.67 | −40.77 | −9.69 | −171 | −0.44 | −7.71 |
| 3 | 66.51 | 66.67 | −39.27 | −9.76 | −165.6 | −0.42 | −6.21 |
| 4 | 68.2 | 61.9 | −39.63 | −11.23 | −163.6 | −0.42 | −6.82 |
| 5 | 65.89 | 61.9 | −39.15 | −12.35 | −166.4 | −0.43 | −6.35 |
| 6 | 64.16 | 61.9 | −37.66 | −10.88 | −161 | −0.41 | −6.58 |
| 7 | 61.44 | 57.14 | −36.11 | −9.68 | −157.7 | −0.41 | −6.6 |
| 8 | 57.77 | 52.38 | −34.01 | −8.19 | −152.7 | −0.4 | −5.61 |
| 9 | 54.43 | 47.62 | −32.41 | −6.72 | −150.3 | −0.4 | −5.61 |
| 10 | 56.01 | 47.62 | −32.77 | −6.69 | −148.3 | −0.39 | −5.99 |
| 11 | 55.29 | 47.62 | −32.16 | −6.7 | −145.7 | −0.38 | −5.73 |
| 12 | 56.23 | 52.38 | −32.54 | −6.73 | −145.9 | −0.38 | −6.72 |
| 13 | 56.94 | 47.62 | −32.52 | −8.27 | −143.7 | −0.37 | −6.82 |
| 14 | 55.63 | 47.62 | −32.42 | −10.3 | −146.7 | −0.38 | −6.35 |
| 15 | 54.69 | 47.62 | −32.04 | −11.42 | −146.5 | −0.38 | −6.58 |
| 16 | 54.69 | 47.62 | −32.04 | −11.16 | −146.5 | −0.38 | −6.22 |
| 17 | 52.16 | 42.86 | −30.85 | −9.98 | −144.5 | −0.38 | −6.59 |
| 18 | 49.58 | 42.86 | −29.19 | −7.71 | −138.4 | −0.37 | −7.07 |
| 19 | 49.74 | 42.86 | −29.26 | −6.21 | −138.5 | −0.37 | −8.64 |
| 20 | 54.17 | 42.86 | −31.53 | −6.82 | −143.9 | −0.38 | −10.65 |
| 21 | 55.42 | 47.62 | −32.64 | −6.35 | −149.1 | −0.39 | −11.77 |
| 22 | 56.34 | 42.86 | −32.88 | −6.58 | −148.2 | −0.39 | −11.4 |
| 23 | 56.13 | 42.86 | −33.25 | −6.6 | −151.7 | −0.4 | −10.2 |
| 24 | 55.05 | 38.1 | −33.24 | −5.61 | −155 | −0.41 | −8.53 |
| 25 | 55.38 | 42.86 | −33.1 | −5.61 | −152.9 | −0.4 | −6.81 |
| 26 | 58.46 | 47.62 | −35.14 | −5.99 | −159.2 | −0.42 | −8.84 |
| 27 | 61.17 | 47.62 | −36.68 | −5.73 | −162.5 | −0.42 | −10.04 |
| 28 | 65.59 | 52.38 | −39.33 | −6.72 | −168.4 | −0.43 | −11.7 |
| 29 | 66.98 | 52.38 | −39.43 | −6.82 | −165.4 | −0.42 | −11.94 |
| 31 | 65.7 | 52.38 | −39.42 | −6.35 | −168.7 | −0.43 | −10.27 |
| 32 | 65.26 | 47.62 | −39.55 | −6.58 | −170.8 | −0.44 | −8.61 |
| 33 | 65.26 | 52.38 | −39.55 | −6.22 | −170.8 | −0.44 | −6.95 |

From the above TABLE 3, it may be noted that RNAi molecules efficient for suppressing or reducing the expression of PMP22 have:
a Tm ranging from about 49.58 to about 56.34° C.,
a ΔH ranging from about 138.4 to about 148.2 kcal/mol,
a ΔS ranging from about 0.37 to about 0.39 kcal/° K/mol,
a GC content ranging from about 42.86 to about 52.38,
a 3′-end stability ranging from about −11.16 to about −6.58 Kcal/mol, and
a 5′-end ΔG ranging from about −11.4 to about −5.73 kcal/mol.

### Example 5: Screening of RNAi Molecules on cPMP22

Further to the identification of particular efficient RNAi molecules on the suppression or reduction of hPMP22 in transfected HEK293 cells, it was intended to evaluate whether those RNAi could be efficient on cynomolgus-PMP22.

It can be advantageous to have a candidate compound for clinical development which can be active on ortholog target protein in cynomolgus, since then this candidate can be tested and developed on this non-human primate model without requiring further adaptation to be translated later on in human clinical development.

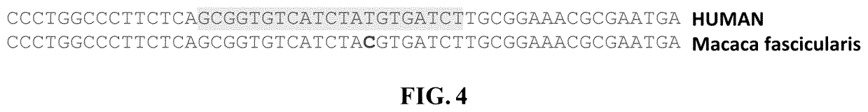

FIG. 4 shows an alignment of sequence between the targeted sequence in exon 5 of hPMP22 and the corresponding sequence in cPMP22. As shown on FIG. 4, the corresponding sequence in cPMP22 is highly homologous to the targeted sequence in exon 5 of hPMP22.

The efficiency of RNAi #11, #12, #16, #17, #18 and #22 molecules was tested on HEK293 cells co-transfected with a cynomolgus-flag vector pCMV3-cPMP22-Flag as above detailed.

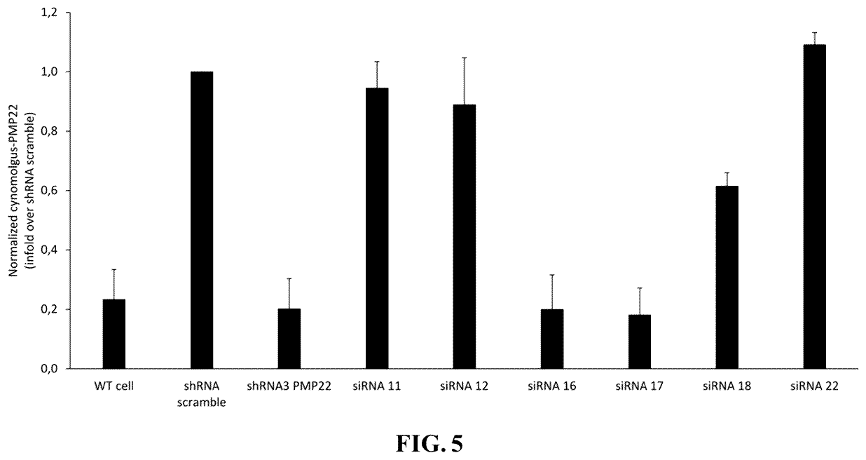

As shown on FIG. 5, co-transfection with scrambled shRNA (control) did not affect the expression of flag-cynomolgus PMP22. The positive control shRNA3 significantly decreased the expression of flag-cynomolgus PMP22 in co-transfected cells to a level similar to the expression level in non-transfected (WT) cells.

Statistically significant decrease of cynomolgus-PMP22-flag expression was observed for the RNAi #16 and #17 after cell co-transfection compared to the negative control. A more moderate decrease in cPMP22 expression was observed with for RNAi #18.

RNAi #16 and #17, and in less extent RNAi #18, are surprisingly and advantageously efficient to reduce or suppress the expression of cPMP22 in transfected HEK293 cells. Therefore, those RNAi molecules can advantageously be used in preclinical development in a cynomolgus model.

### Example 6: AAV9-Expressing RNAi Molecules on HEK293-Expressing hPMP22 or cPMP22

In this set of experiments, efficiency of RNAi molecules #16 and #17 were validated in AAV9-vector on HEK293 cells expressing human (hPMP22) or cynomolgus (cPMP22) PMP22 protein.

In this set of experiments, the HEK293 cells were transfected with human-flag vector pCMV3-cPMP22-Flag or a cynomolgus-flag vector pCMV3-cPMP22-Flag using Lipofectamin™ 200 reagent. Then, the cells were infected with increasing concentrations of AAV9-vectors containing an RNAi molecule (#16 or #17) or a shRNA scramble (control), and the total PMP22 expression was analyzed by Western blotting.

The transfection of HEK293 cells with 750 ng of cynomolgus or human flag-PMP22 led to a quantifiable PMP22 expression. As expected, no significant differences were observed in the expression of flag-PMP22 proteins 48 hours after AAV9.shRNA scramble (control) infection at the tested doses.

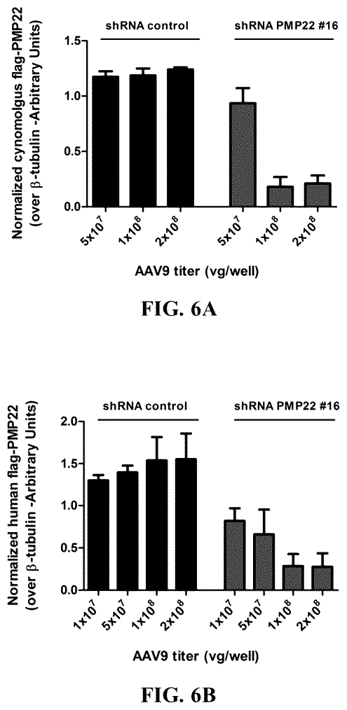

However, a significant decrease of cynomolgus and human flag-PMP22 expression was observed when the transfected cells were infected with 5×10⁷ vg/well, 1×10⁸ vg/well and 2×10⁸ vg/well of AAV9.RNAi #16, compared to the shRNA control (FIGS. 6A and 6B).

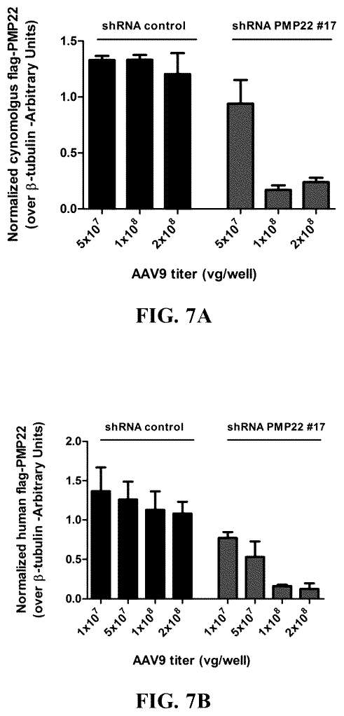

Similar results were observed between shRNA PMP22 #16 and #17. A significant decrease of cynomolgus and human flag-PMP22 expression was observed when the transfected cells were infected with, 1×10⁸ vg/well and 2×10⁸ vg/well of AAV9.RNAi #17, compared to the shRNA control (FIGS. 7A and 7B).

Interestingly, whereas the RNAi #16 led to a decrease of flag-PMP22 when cells were infected with 5×10⁷ vg/well, no statistical difference was observed using RNAi #17 at the same dose. This difference could be explained by a lower affinity of the RNAi #17 compared to the RNAi #16 because of the differences of nucleic acid sequence.

In conclusion, significant decrease of cynomolgus and human flag-PMP22 expression was observed when HEK293 transfected cells were infected with 5×10⁷ vg/well, 1×10⁸ vg/well and 2×10⁸ vg/well of AAV9.RNAi #16, and with 1×10⁸ vg/well and 2×10⁸ vg/well of AAV9.RNAi #17 respectively.

These results confirm that both RNAi #16 and #17 are able to reduce the expression of both hPMP22 and cPMP22 proteins in vitro. Therefore, those RNAi molecules reveal themselves as interesting lead candidate for further clinical development of a gene therapy, based on an adeno-associated vector, for treating or preventing the CMT1A or 1E diseases.

### Example 7: Viral Particle Production

### Materials and Methods

ShRNAs targeting human and cynomolgus PMP22 mRNA were cloned in pAAV vector. Vector production was performed by Centre de Production de Vecteurs facility of INSERM UMR 1089, IRS 2 Nantes Biotech—Université de Nantes. Briefly, recombinant AAVs were manufactured by co-transfection of HEK293 cells with pAAV and pDP9 plasmids and purified by cesium chloride density gradients followed by extensive dialysis against phosphate-buffered saline (PBS). Vector titers were determined by qPCR, the target amplicons correspond to the inverted terminal repeat (ITR) sequences, ITR-2.

### Results

As shown on FIG. 8, production of AAV9 viral particles expressing RNAi #16 is reduced compared to AAV9 viral particles expressing RNAi #17 or control AAV9 viral particles.

Thus, it was observed that adeno-associated vector production yield was much higher for RNAi #17 compared to RNAi #16, giving to this compound a further advantage in terms of industrial production.

### Example 8: Effect of AAV9 Vector Expressing RNAi #17 on Mouse Model of CMT1A

### Materials and Methods

### Mice

C3 CMT1A humanized mice, overexpressing human PMP22, were housed in macrolon cages (UniqUse, Ref. M.BTM) with filter hoods, in a room where the air is continuously filtered, thereby avoiding contamination. During experiments, paired animals were caged at constant temperature with a day/night cycle of 12/12 hours. Animals received water (control tap water) and nutrition ad libitum.

Animal protocol has been approved by the Animal Studies Committee of Languedoc Roussillon. This protocol and the laboratory procedures comply with French legislation, which implements the European Directives (Reference Number: D3417223, APAFIS #23920-2020020320279696 v3). Animal health was examined every day to ensure that only animals in good health enter the testing procedures and follow up the study.

Control AAV9 or AAV9 vector expressing RNAi #17 at three different doses (4.2×10⁹ vg/nerve, 1.7×10¹⁰ vg/nerve and 3.4×10¹⁰ vg/nerve) were administrated through a single bilateral intraneural injection (intrafascicular) in mice sciatic nerves 4 days after birth.

TABLE 4

| | | | | | Clinical | WB |
|---|---|---|---|---|---|---|
| | | Dose | | | analysis | analysis |
| | Com- | vg/ | Mouse | # of | Age in | Age in |
| Group | pound | nerve | strain | animals | months | months |
| Non-injected | NA | NA | C3 | 6 | 1 and 2 | 2 |
| control | | | | | | |
| Control | AAV9- | 3.4 × | C3 | 6 | 1 and 2 | 2 |
| AAV9 | RNAi | 10¹⁰ | | | | |
| | scramble | | | | | |
| AAV9-sh17 | AAV9- | 4.2 × | C3 | 7-6 | 1 and 2 | 2 |
| 4.2 × 10⁹ | RNAi#17 | 10⁹ | | | | |
| vg/nerve | | | | | | |
| AAV9-sh17 | AAV9- | 1.7 × | C3 | 6 | 1 and 2 | 2 |
| 1.7 × 10¹⁰ | RNAi#17 | 10¹⁰ | | | | |
| vg/nerve | | | | | | |
| AAV9-sh17 | AAV9- | 3.4 × | C3 | 7-6 | 1 and 2 | 2 |
| 3.4 × 10¹⁰ | RNAi#17 | 10¹⁰ | | | | |
| vg/nerve | | | | | | |

### Rotarod

A rotating rod apparatus (Bioseb, Ref. Bx-rod-m) was used to measure walking performances, coordination and balance. Mice were first given a 1-days pretraining trial to familiarize them with the rotating rod. Latency to fall was measured at a successively increased speed from 4 to 40 rpm over a 300-second maximum time period. Each animal underwent 3 trials a day. For each day, values from the 3 trials were averaged for each animal, and then averaged for each group.

### Grip Test

Neuromuscular strengths of all mice were assessed in standardized grip strength tests for all limbs (Bioseb, Ref. BIO-GS3). All limb grip strength was measured by supporting the limbs on a horizontal grid connected to a gauge and pulling the animal's tail. The maximum force (measured in newtons) exerted on the grid before the animal lost its grip was recorded, and the mean of 3 repeated measurements was calculated. Finally, the data were averaged for each treated group.

### Sciatic Nerve Electrophysiology

Standard electromyography was performed on mice anesthetized with ketamine/xylazine mixture following SOP-A15-V1. A pair of steel needle electrodes (AD Instruments, MLA1302) were placed subcutaneously along the nerve at the sciatic notch (proximal stimulation). A second pair of electrodes were placed along the tibial nerve above the ankle (distal stimulation). Supramaximal square-wave pulses, lasting 10 ms at 1 mA were delivered using a PowerLab 26T (AD Instruments). Compound muscle action potential (CMAP) was recorded from the intrinsic foot muscles using steel electrodes. Both amplitudes and latencies of CMAP were determined. The distance between the 2 sites of stimulation was measured alongside the skin surface with fully extended legs, and nerve conduction velocities (NCVs) were calculated automatically from sciatic nerve latency measurements using Excel.

### Western-Blotting

2 months postnatal, after electrophysiology analysis and animal sacrifice, the left sciatic nerve of all animals were sampled, solubilized in 4° C. lysis buffer (0.5 mL Tris HCl 1 M pH8, 0.375 mL NaCl 4 M, 40 μl EDTA 0.5M, 100 μl Triton and 8.9 mL H2O) completed with protease inhibitors (Fisher Scientific, France). Each nerve was cut in a small parts (around 0.5 or 1 mm each part) sonicated three times during 10 s on ice (Microson ultrasonic cell disruptorXL, Microsonic), vortexed 5 times during 2 min and in rotation over night at 4° C. Then, centrifugated at 12000 rpm during 30 min at 4° C. and supernatant stored at −20° C. before protein analysis. Then, ten micrograms of protein samples were denatured using Laemmli/0-mercaptoethanol (Merck, S3401) incubation at 95° C. during 10 minutes, separated on a denaturing 10% SDS-polyacrylamide gel and transferred onto nitrocellulose membranes. Membranes were blocked for 60 min with 5 mL of Licor Blocking Buffer. The following primary antibodies were incubated overnight at 4° C. in the same blocking buffer: rabbit anti-PMP22 (1:100, abcam, Ref. 211052) and mouse anti-b-tubulin (1:500, Sigma Aldrich, T8578-100 μL). The following day the membranes were washed 3 times for 10 minutes in TBS Tween-20 (0.1% V/V) and then incubated for 1 hour at room temperature with the secondary fluorescence antibodies: goat anti-mouse IRDye 800 (1:10.000, LI-COR Biosciences, Ref 926-32210) and donkey anti-rabbit IRDye 680 (1:10.000, LI-COR Biosciences, Ref 926-68073). After secondary antibody incubation, the membranes were washed three times for 10 min with TBS Tween-20 (0.1% V/V). Samples were leaded randomly to avoid membrane variability. For loading control b-tubulin expression was detected using a mouse anti-b-tubulin and a goat anti-mouse IRDye 800 (red fluorescence). Visualization of the bands was performed using Odyssey CLX LI-COR Imaging System in green and red fluorescences and band quantification was performed using Image J software (version 4.0). PMP22 bands fluorescence intensities were normalized on the respective fluorescence band of b-tubulin to obtain “Normalized PMP22/b-tubulin” values that are plotted in the graphs in arbitrary units.

### Results

The aim of this study was to evaluate the efficacy of the AAV9 vector expressing RNAi #17 in the C3 CMT1A humanized mouse model, which overexpresses human PMP22. AAV9-sh17 was administered at three different doses administrated after a single bilateral intraneural injection (intrafascicular) in sciatic nerves 4 days after birth, and compared to a control AAV9 vector. AAV9-sh17 efficacy was evaluated 1) biochemically using a Western blot analysis of PMP22 expression in sciatic nerves (2 months after injection), 2) electrophysiologically measuring nerve conduction velocity and compound of muscular action potential (CMAP) in sciatic nerves (1 and 2 months after injections) and clinically using Rotarod and grip test (1 and 2 months after injections). Parameters measured at 1 month after injection were used as baseline for repeated measures as the symptoms of the disease appear later.

### Western-Blot Analysis

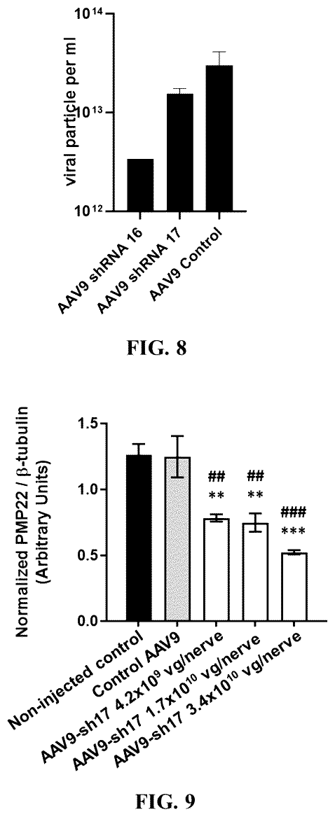

As shown on FIG. 9, a significant decrease of PMP22 was induced by AAV9 vector expressing RNAi #17 at all doses (4.2×10⁹ vg/nerve, 1.7×10¹⁰ vg/nerve and 3.4×10¹⁰ vg/nerve), as compared to non-injected animals and animals treated with a control AAV9 vector.

These results demonstrate that AAV9 vector expressing RNAi #17 is effective to decrease human PMP22 expression in mouse expressing human PMP22.

### Rotarod

At 1 month old, before the reported onset of the disease, similar rotarod latencies were observed for the five groups of animals (data not shown). This time point was used as the baseline for all groups in repeated measurements.

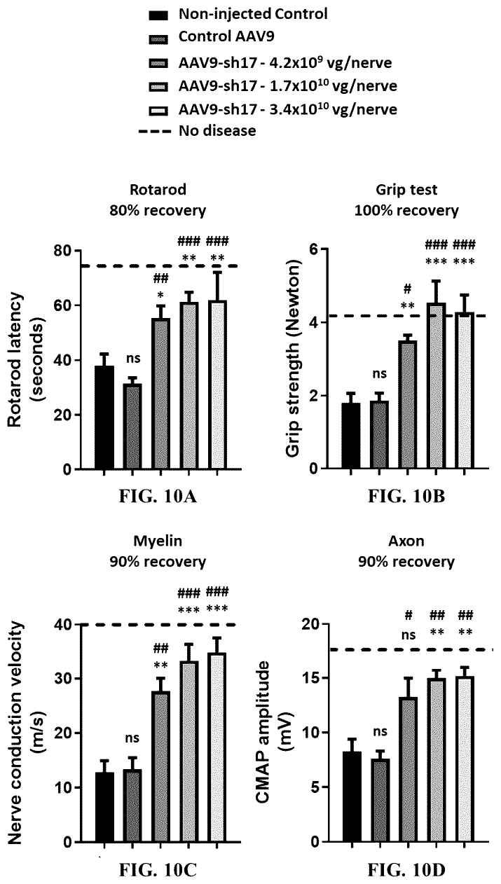

At 2 months old, after the reported onset of the disease, a significant decrease of the rotarod latency was observed in non-injected animals and in animals injected with the control AAV9 vector as compared to baseline (FIG. 10A, baseline represented by the dashed line). A significant increase of the rotarod latency was observed for the animals injected with AAV9 vector expressing RNAi #17 at all three doses, as compared to non-injected animals and animals injected with the control AAV9 vector (FIG. 10A).

### Grip Test

At 1 month old, before the reported onset of the disease, similar grip strengths were observed for the five groups of animals (data not shown). This time point was used as the baseline for all groups in repeated measurements.

At 2 months old, after the reported onset of the disease, a significant decrease of the grip strength was observed in non-injected animals and in animals injected with the control AAV9 vector as compared to baseline (FIG. 10B, baseline represented by the dashed line). A significant increase was observed for the animals injected with AAV9 vector expressing RNAi #17 at all three doses, as compared to non-injected animals and animals injected with the control AAV9 vector (FIG. 10B).

### Nerve Conduction Velocity

Similar nerve conduction velocities (NCVs) were observed at 1 month old for the five groups of animals (data not shown). These data served as a baseline for repeated measures at 2 months.

At 2 months, a significant decrease of the NCV was observed in the NCV of non-injected animals and of animals injected with the control AAV9 vector as compared to baseline (FIG. 10C, baseline represented by the dashed line). At this age, a significant increase of the NCV was observed in animals injected with AAV9 vector expressing RNAi #17 at all three doses, as compared to non-injected animals and animals injected with the control AAV9 vector (FIG. 10C). This increase was also dose dependent as the AAV9 vector expressing RNAi #17 1.7×10¹⁰ vg/nerve and 3.4×10¹⁰ vg/nerve dose groups showed a significant increase in NCV compared to the AAV9 vector expressing RNAi #17 4.2×10⁹ vg/nerve dose group.

### CMAP Amplitude

Similar CMAPs were observed at the baseline (1 month old) for the five groups of animals (data not shown).

A significant decrease of the CMAP was observed in the control AAV9 group of animals at 2 months old compared to the non-injected control group of animals (FIG. 10D).

Moreover, significant increase of the CMAP was observed in the three AAV9 vector expressing RNAi #17 treated groups at 2 months old compared to the control AAV9 group (FIG. 10D). This increase was also dose-dependent confirming a direct efficacy of the AAV9 vector expressing RNAi #17 on the muscular impairment induced by the CMT1A phenotype.

### Conclusions

Neuromuscular impairment characterized by a decrease of the rotarod latency and grip strength and decrease of nerve conduction amplitude and velocity were observed in the preclinical CMT1A mouse model non-injected and injected with AAV9-RNAi scramble (control AAV9) at two months old.

The AAV9-RNAi #17 treated group presented a significant increase of the rotarod latency, grip strength and nerve conduction amplitude and velocity at two months old compared the AAV9-RNAi scramble group (control AAV9).

Moreover, the three AAV9-RNAi #17 treated groups also presented a decrease of the PMP22 protein in the sciatic nerve of C3 mice confirming the molecular efficacy of the viral vectors when injected at 4.2×10⁹ vg/nerve, 1.7×10¹⁰ vg/nerve and 3.4×10¹⁰ vg/nerve respectively.

Taken together, this study confirms that intranerve injection of the AAV9 vector expressing RNAi #17 presents significant protective efficacy on CMT1A neuropathy targeting PMP22 overexpression in Schwann cells by increasing neuromuscular and electrophysiological performances in two-month old C3 mice.

### Example 9: Effect of AAV9 Vector Expressing RNAi #17 on Cynomolgus Monkeys

### Materials and Methods

### Cynomolgus Monkeys

Control AAV9 or AAV9 vector expressing RNAi #17 were delivered bilaterally at two different doses in sciatic nerves of 3 adult cynomolgus monkeys through intraneural perifascicular (INPF) injections, as indicated in TABLE 4. PMP22 expression level was measured in treated sciatic nerves one month after injections using Western blotting.

TABLE 5

| | | | # of | |
|---|---|---|---|---|
| | | | sciatic | |
| | | | nerves | |
| | | Injected | (left & | Sampling |
| Group | Injection | titer | right) | time |
| Animal | AAV2/9 | 5 × 10¹² | 2 | 29 days post |
| control | CMV-GFP | vg/nerve | | injection |
| (negative | | | | |
| control) | | | | |
| Animal 1 | AAV9- | 2 × 10¹² | 2 | 28 days post |
| | RNAi #17 | vg/nerve | | injection |
| Animal 2 | AAV9- | 5 × 10¹² | 2 | 28 days post |
| | RNAi #17 | vg/nerve | | injection |
| Animal 3 | AAV9- | 5 × 10¹² | 2 | 28 days post |
| | RNAi #17 | vg/nerve | | injection |

### Western Blotting

Sciatic nerve of all animals were sampled, solubilized in 4° C. lysis buffer (0.5 mL Tris HCl 1 M pH8, 0.375 mL NaCl 4 M, 40 μl EDTA 0.5M, 100 μl Triton and 8.9 mL H2O) completed with protease inhibitors (Fisher Scientific, France). Each nerve was cut in a small parts (around 0.5 or 1 mm each part) sonicated three times during 10 seconds on ice (Microson ultrasonic cell disruptorXL, Microsonic), vortexed 5 times during 2 min and in rotation over night at 4° C. Then, ten micrograms of protein samples were denatured using Laemmli/β-mercaptoethanol (Merck, S3401) incubated at 95° C. during 10 minutes, separated on a denaturing 10% SDS-polyacrylamide gel and transferred onto nitrocellulose membranes. Membranes were blocked for 60 min with 5 mL of Licor Blocking Buffer. The following primary antibodies were incubated overnight at 4° C. in the same blocking buffer: rabbit anti-PMP22 (1:100, abcam, Ref. 211052) and mouse anti-b-tubulin (1:500, Sigma Aldrich, T8578-100 μL). The following day the membranes were washed 3 times for 10 minutes in TBS Tween-20 (0.1% V/V) and then incubated for 1 hour at room temperature with the secondary fluorescence antibodies: goat anti-mouse IRDye 800 (1:10.000, LI-COR Biosciences, Ref 926-32210), and donkey anti-rabbit IRDye 680 (1:10.000, LI-COR Biosciences, Ref 926-68073). After secondary antibody incubation, the membranes were washed three times for 10 min with TBS Tween-20 (0.1% V/V). Visualization of the bands was performed using Odyssey CLX LI-COR Imaging System and band quantification was performed using Image J software (version 4.0). The PMP22 concentration was normalized over β-tubulin concentration considered as control protein (constant housekeeping protein).

### Results

The aim of this study was to evaluate the effect of AAV9 vector expressing RNAi #17 on PMP22 expression level in sciatic nerves of cynomolgus monkeys. Control AAV9 or AAV9 vector expressing RNAi #17 at two different doses were delivered bilaterally in the sciatic nerves of 3 adult cynomolgus monkeys through intraneural perifascicular (INPF) injections.

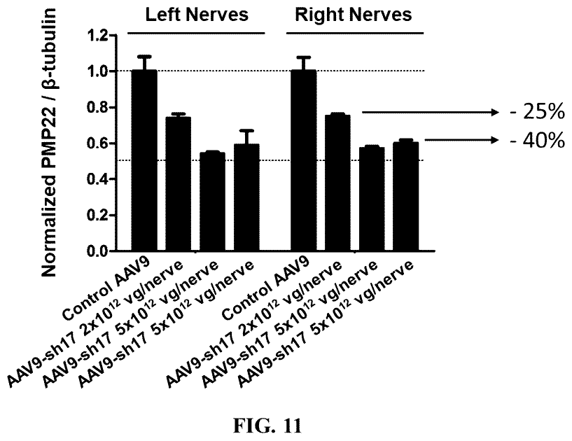

As shown on FIG. 11, Western blot analysis of sciatic nerves (left and right) of cynomolgus monkeys injected with AAV9 vector expressing RNAi #17 demonstrated that AAV9 vector expressing RNAi #17 was able to reduce the concentration of PMP22 at an infold of 0.75 and 0.57 compared to the negative control group when injected at 2×10¹² vg/nerve and at 5×10¹² vg/nerve respectively.

In conclusion, it was surprisingly found by the inventors that, among the 33 tested RNAi molecules targeting exon 5 of the PMP22 protein, the particular RNAi #17 (having the antisense sequence of SEQ ID NO: 61) was the only RNAi molecule that:
efficiently suppressed or reduced the expression of hPMP22 in its full-length as well as N-terminally truncated forms,
efficiently reduced or suppressed the expression of cynomolgus PMP22,
presented a high adeno-associated vector yield production,
presented significant protective efficacy on CMT1A neuropathy targeting PMP22 overexpression in Schwann cells by increasing neuromuscular and electrophysiological performances in a mouse model of CMT1A, and
was able to reduce PMP22 expression level in sciatic nerves of cynomolgus monkeys, following bilaterally delivery in the sciatic nerves of adult cynomolgus monkeys through intraneural perifascicular (INPF) injections.

### REFERENCES

Ayuso, E., Mingozzi, F. & Bosch, F. Production, purification and characterization of adeno-associated vectors. CGT 10, 423-436 (2010)
Baichwal and Sugden, “Vectors for gene transfer derived from animal DNA viruses: Transient and stable expression of transferred genes,” In: Kucherlapati R, ed. Gene transfer. New York: Plenum Press, 117-148(1986)
Bass B L. RNA interference. The short answer. Nature. 2001 May 24; 411(6836):428-9. doi: 10.1038/35078175. PMID: 11373658.
Boutary S, Caillaud M, El Madani M, Vallat J M, Loisel-Duwattez J, Rouyer A, Richard L, Gracia C, Urbinati G, Desmaële D, Echaniz-Laguna A, Adams D, Couvreur P, Schumacher M, Massaad C, Massaad-Massade L. Squalenoyl siRNA PMP22 nanoparticles are effective in treatin mouse models of Charcot-Marie-Tooth disease type 1 A. Commun Biol. 2021 Mar. 9; 4(1):317. doi: 10.1038/s42003-021-01839-2. PMID: 33750896.
Breslauer, K. J., Frank, R., Blocker, H., & Marky, L. A. (1986) Proc. Natl. Acad. Sci. U.S.A. 83, 3746-3750
Freier, S. M., Kierzek, R., Jaeger, J. A., Sugimoto, N., Caruthers, M. H., Neilson, T., & Turner, D. H. (1986) Proc. Natl. Acad. Sci. U.S.A. 83, 9373-9377
Brummelkamp T R, Bernards R, Agami R. A system for stable expression of short interfering RNAs in mammalian cells. Science. 2002 Apr. 19; 296(5567):550-3. doi: 10.1126/science.1068999. Epub 2002 Mar. 21. PMID: 11910072.
Carter B J. Adeno-associated virus vectors. Cuff Opin Biotechnol. 1992 October; 3(5):533-9. doi: 10.1016/0958-1669(92)90082-t. PMID: 1369403.
Chambers R, Gillespie G Y, Soroceanu L, Andreansky S, Chatterjee S, Chou J, Roizman B, Whitley R J. Comparison of genetically engineered herpes simplex viruses for the treatment of brain tumors in a scid mouse model of human malignant glioma. Proc Natl Acad Sci USA. 1995 Feb. 28; 92(5):1411-5. doi: 10.1073/pnas.92.5.1411. PMID: 7877992; PMCID: PMC42529.
Coupar B E, Andrew M E, Boyle D B. A general method for the construction of recombinant vaccinia viruses expressing multiple foreign genes. Gene. 1988 Aug. 15; 68(1):1-10. doi: 10.1016/0378-1119(88)90593-8. PMID: 2851485.
Davis et al. (1986) Basic Methods in Molecular Biology, Elsevier
Dong Y, Siegwart D J, Anderson D G. Strategies, design, and chemistry in siRNA delivery systems. Adv Drug Deliv Rev. 2019; 144:133-147. doi:10.1016/j.addr.2019.05.004
Elbashir S M, Harborth J, Lendeckel W, Yalcin A, Weber K, Tuschl T. Duplexes of 21-nucleotide RNAs mediate RNA interference in cultured mammalian cells. Nature. 2001 May 24; 411(6836):494-8. doi: 10.1038/35078107. PMID: 11373684.
Elbashir S M, Martinez J, Patkaniowska A, Lendeckel W, Tuschl T. Functional anatomy of siRNAs for mediating efficient RNAi in Drosophila melanogaster embryo lysate. EMBO J. 2001 Dec. 3; 20(23):6877-88. doi: 10.1093/emboj/20.23.6877. PMID: 11726523; PMCID: PMC125328.
Gautier, B., Hajjar, H., Soares, S. et al. AAV2/9-mediated silencing of PMP22 prevents the development of pathological features in a rat model of Charcot-Marie-Tooth disease 1 A. Nat Commun 12, 2356 (2021). https://doi.org/10.1038/s41467-021-22593-3
Hannon G J. RNA interference. Nature. 2002 Jul. 11; 418(6894):244-51. doi: 10.1038/418244a. PMID: 12110901.
Hope M J, Mui B, Ansell S, Ahkong Q F. Cationic lipids, phosphatidylethanolamine and the intracellular delivery of polymeric, nucleic acid-based drugs (review). Mol Membr Biol. 1998 January-March; 15(1):1-14. doi: 10.3109/09687689809027512. PMID: 9595549.
Jerath N U, Shy M E. Hereditary motor and sensory neuropathies: Understanding molecular pathogenesis could lead to future treatment strategies. Biochim Biophys Acta. 2015; 1852(4):667-678. doi:10.1016/j.bbadis.2014.07.031
Johnson J S, Roux K J, Fletcher B S, Fortun J, Notterpek L. Molecular alterations resulting from frameshift mutations in peripheral myelin protein 22: implications for neuropathy severity. J Neurosci Res. 2005 Dec. 15; 82(6):743-52. doi: 10.1002/jnr.20691. PMID: 16273544.
Kotin R M. Prospects for the use of adeno-associated virus as a vector for human gene therapy. Hum Gene Ther. 1994 July; 5(7):793-801. doi: 10.1089/hum.1994.5.7-793. PMID: 7981305.
Li J, Parker B, Martyn C, Natarajan C, Guo J. The PMP22 gene and its related diseases. Mol Neurobiol. 2013; 47(2):673-698. doi:10.1007/s12035-012-8370-x
Liao, Y C., Tsai, P C., Lin, T S. et al. Clinical and Molecular Characterization of PMP22 point mutations in Taiwanese patients with Inherited Neuropathy. Sci Rep 7, 15363 (2017). https://doi.org/10.1038/s41598-017-14771-5
Lebkowski J S, McNally M M, Okarma T B, Lerch L B. Adeno-associated virus: a vector system for efficient introduction and integration of DNA into a variety of mammalian cell types. Mol Cell Biol. 1988 October; 8(10):3988-96. doi: 10.1128/mcb.8.10.3988-3996.1988. PMID: 2847025; PMCID: PMC365467.
Lewis J G, Lin K Y, Kothavale A, Flanagan W M, Matteucci M D, DePrince R B, Mook R A Jr, Hendren R W, Wagner R W. A serum-resistant cytofectin for cellular delivery of antisense oligodeoxynucleotides and plasmid DNA. Proc Natl Acad Sci USA. 1996 Apr. 16; 93(8):3176-81. doi: 10.1073/pnas.93.8.3176. PMID: 8622909; PMCID: PMC39578.
McCarty D M, Christensen M, Muzyczka N. Sequences required for coordinate induction of adeno-associated virus p19 and p40 promoters by Rep protein. J Virol. 1991 June; 65(6):2936-45. doi: 10.1128/JVI.65.6.2936-2945.1991. PMID: 2033660; PMCID: PMC240929.
McLaughlin S K, Collis P, Hermonat P L, Muzyczka N. Adeno-associated virus general transduction vectors: analysis of proviral structures. J Virol. 1988 June; 62(6):1963-73. doi: 10.1128/JVI.62.6.1963-1973.1988. PMID: 2835501; PMCID: PMC253280.
McManus M T, Petersen C P, Haines B B, Chen J, Sharp P A. Gene silencing using micro-RNA designed hairpins. RNA. 2002 June; 8(6):842-50. doi: 10.1017/s1355838202024032. PMID: 12088155; PMCID: PMC1370301.
Mann R, Mulligan R C, Baltimore D. Construction of a retrovirus packaging mutant and its use to produce helper-free defective retrovirus. Cell. 1983 May; 33(1):153-9. doi: 10.1016/0092-8674(83)90344-6. PMID: 6678608.
Martinez J, Patkaniowska A, Urlaub H, Lührmann R, Tuschl T. Single-stranded antisense siRNAs guide target RNA cleavage in RNAi. Cell. 2002 Sep. 6; 110(5):563-74. doi: 10.1016/s0092-8674(02)00908-x. PMID: 12230974.
McIntyre G J, Fanning G C. Design and cloning strategies for constructing shRNA expression vectors. BMC Biotechnol. 2006; 6:1. Published 2006 Jan. 5. doi:10.1186/1472-6750-6-1
Muzyczka N. Use of adeno-associated virus as a general transduction vector for mammalian cells. Curr Top Microbiol Immunol. 1992; 158:97-129. doi: 10.1007/978-3-642-75608-5_5. PMID: 1316261.
Nicolas and Rubinstein “Retroviral vectors,” In: Vectors: A survey of molecular cloning vectors and their uses, Rodriguez and Denhardt (eds.), Stoneham: Butterworth, 494-513 (1988)
Nicolau C, Sene C. Liposome-mediated DNA transfer in eukaryotic cells. Dependence of the transfer efficiency upon the type of liposomes used and the host cell cycle stage. Biochim Biophys Acta. 1982 Oct. 11; 721(2):185-90. doi: 10.1016/0167-4889(82)90067-2. PMID: 6982727.
Nicolau C, Legrand A, Grosse E. Liposomes as carriers for in vivo gene transfer and expression. Methods Enzymol. 1987; 149:157-76. doi: 10.1016/0076-6879(87)49054-x. PMID: 3320679.
Nykanen A, Haley B, Zamore PD. ATP requirements and small interfering RNA structure in the RNA interference pathway. Cell. 2001 Nov. 2; 107(3):309-21. doi: 10.1016/s0092-8674(01)00547-5. PMID: 11701122.
Pillai-Kastoori, L., Schutz-Geschwender, A. R. & Harford, J. A. A systematic approach to quantitative Western blot analysis. Anal. Biochem. 593, 113608 (2020).
Puhlmann M, Gnant M, Brown C K, Alexander H R, Bartlett D L. Thymidine kinase-deleted vaccinia virus expressing purine nucleoside phosphorylase as a vector for tumor-directed gene therapy. Hum Gene Ther. 1999 Mar. 1; 10(4):649-57. doi: 10.1089/10430349950018724. PMID: 10094208.
REMINGTON's PHARMACEUTICAL SCIENCES, 18th Edition, (A. R. Gennaro, ed.), 1990, Mack Publishing Company
Ridgeway, “Mammalian expression vectors,” In: Vectors: A survey of molecular cloning vectors and their uses. Rodriguez and Denhardt, eds. Stoneham: Butterworth, 467-492(1988)
Riordan J R, Rommens J M, Kerem B, Alon N, Rozmahel R, Grzelczak Z, Zielenski J, Lok S, Plavsic N, Chou J L, et al. Identification of the cystic fibrosis gene: cloning and characterization of complementary DNA. Science. 1989 Sep. 8; 245(4922):1066-73. doi: 10.1126/science.2475911. Erratum in: Science 1989 Sep. 29; 245(4925):1437. PMID: 2475911.
Sambrook et al., 2001, MOLECULAR CLONING: A LABORATORY MANUAL, 3d ed., Cold Spring Harbor Laboratory Press, Cold Spring Harbor, N.Y.
Schwarz D S, Hutvgner G, Haley B, Zamore P D. Evidence that siRNAs function as guides, not primers, in the Drosophila and human RNAi pathways. Mol Cell. 2002 September; 10(3):537-48. doi: 10.1016/s1097-2765(02)00651-2. PMID: 12408822.
Serfecz J, Bazick H, Al Salihi M O, Turner P, Fields C, Cruz P, Renne R, Notterpek L. Downregulation of the human peripheral myelin protein 22 gene by miR-29a in cellular models of Charcot-Marie-Tooth disease. Gene Ther. 2019 December; 26(12):455-464. doi: 10.1038/s41434-019-0098-z. Epub 2019 Aug. 27. PMID: 31455873; PMCID: PMC6920087.
Shelling A N, Smith M G. Targeted integration of transfected and infected adeno-associated virus vectors containing the neomycin resistance gene. Gene Ther. 1994 May; 1(3):165-9. PMID: 7584077.
Srivastava C H, Samulski R J, Lu L, Larsen S H, Srivastava A. Construction of a recombinant human parvovirus B19: adeno-associated virus 2 (AAV) DNA inverted terminal repeats are functional in an AAV-B19 hybrid virus. Proc Natl Acad Sci USA. 1989 October; 86(20):8078-82. doi: 10.1073/pnas.86.20.8078. PMID: 2554300; PMCID: PMC298218.
Thimmappaya B, Weinberger C, Schneider R J, Shenk T. Adenovirus VAI RNA is required for efficient translation of viral mRNAs at late times after infection. Cell. 1982 December; 31(3 Pt 2):543-51. doi: 10.1016/0092-8674(82)90310-5. PMID: 6297772.
Tuschl T, Zamore P D, Lehmann R, Bartel D P, Sharp P A. Targeted mRNA degradation by double-stranded RNA in vitro. Genes Dev. 1999 Dec. 15; 13(24):3191-7. doi: 10.1101/gad.13.24.3191. PMID: 10617568; PMCID: PMC317199.U.S. Pat. No. 5,139,941—AAV transduction vectors
U.S. Pat. No. 4,861,719—DNA constructs for retrovirus packaging cell lines
U.S. Pat. No. 5,139,941—AAV transduction vectors
U.S. Pat. No. 5,173,414—Production of recombinant adeno-associated virus vectors
U.S. Pat. No. 5,278,056—Retroviral packaging cell lines and process of using same
U.S. Pat. No. 5,767,099—Cationic amphiphiles containing amino acid or derivatized amino acid groups for intracellular delivery of therapeutic molecules
U.S. Pat. No. 5,780,053—Cationic phospholipids for transfection
U.S. Pat. No. 5,830,430—Cationic lipids and the use thereof
U.S. Pat. No. 5,851,548—Liposomes containing cationic lipids and vitamin D
U.S. Pat. No. 5,855,910—Cationic phospholipids for transfection
U.S. Pat. No. 5,882,877—Adenoviral vectors for gene therapy containing deletions in the adenoviral genome
U.S. Pat. No. 6,013,516—Vector and method of use for nucleic acid delivery to non-dividing cells
U.S. Pat. No. 6,506,559—Genetic inhibition by double-stranded RNA
U.S. Pat. No. 6,573,099—Genetic constructs for delaying or repressing the expression of a target gene
WO 1992/1070 A1—High efficiency packaging of mutant adeno-associated virus using amber suppressions
WO 1993/03769 A1—Adenovirus mediated transfer of genes to the gastrointestinal tract
WO 1994/19478—Production of high titer helper-free retroviruses by transient transfection
WO 1995/14785—Method for producing recombinant virus
WO 1996/22378—Cells for the production of recombinant adenoviruses
WO 1999/07409—Product comprising at least a double stranded RNA combined with at least an antiviral agent
WO 1999/32619—Genetic inhibition by double-stranded RNA
WO 2000/44895—Method and medicament for inhibiting the expression of a given gene
WO 00/44914—Composition and method for in vivo and in vitro attenuation of gene expression using double stranded RNA
WO 2001/29058—RNA interference pathway genes as tools for targeted genetic interference
WO 2001/36646—Inhibiting Gene Expression with dsRNA
WO 2001/68836—Methods and compositions for RNA interference
WO 2017/005806—Methods and Pharmaceutical Compositions for Expressing a Polynucleotide of Interest in the Peripheral Nervous System of a Subject
WO 2020/064749—Antisense Rna Targeting Pmp22 for the Treatment of Charcot-Marie-Tooth la Disease
Zhao H T, Damle S, Ikeda-Lee K, Kuntz S, Li J, Mohan A, Kim A, Hung G, Scheideler M A, Scherer S S, Svaren J, Swayze E E, Kordasiewicz H B. PMP22 antisense oligonucleotides reverse Charcot-Marie-Tooth disease type 1A features in rodent models. J Clin Invest. 2018 Jan. 2; 128(1):359-368. doi: 10.1172/JCI96499. Epub 2017 Dec. 4. PMID: 29202483; PMCID: PMC5749515
Zhou S Z, Cooper S, Kang L Y, Ruggieri L, Heimfeld S, Srivastava A, Broxmeyer H E. Adeno-associated virus 2-mediated high efficiency gene transfer into immature and mature subsets of hematopoietic progenitor cells in human umbilical cord blood. J Exp Med. 1994 Jun. 1; 179(6):1867-75. doi: 10.1084/jem.179.6.1867. PMID: 7515101; PMCID: PMC2191525.

1-11. (canceled)

12. An adeno-associated virus (AAV) vector comprising an RNAi molecule comprising an antisense oligonucleotide of sequence SEQ ID NO: 61, and/or an oligonucleotide coding for an antisense oligonucleotide of sequence SEQ ID NO: 61.

13. The AAV vector according to claim 12, wherein said RNAi molecule is a shRNA or a miRNA.

14. The AAV vector according to claim 12, wherein said RNAi molecule inhibits a PMP22 protein expression and/or activity.

15. The AAV vector according to claim 12, wherein said AAV vector is selected from the group consisting of AAV9, AAV2/9, AAV10, AAVrh10 and AAV2/rh10.

16. The AAV vector according to claim 12, wherein said AAV vector is an AAV serotype 9 (AAV9).

17. The AAV vector according to claim 12, wherein said AAV vector is a single-stranded AAV or a self-complementary AAV.

18. An isolated host cell containing an AAV vector according to claim 12.

19. A pharmaceutical composition comprising an AAV vector according to claim 12, and a pharmaceutically acceptable excipient.

20. A method of treating a patient in need thereof, said method comprising administering to said patient a therapeutically effective amount of an AAV vector according to claim 12, or a pharmaceutical composition comprising said AAV vector and a pharmaceutically acceptable excipient.

21. A method of preventing and/or treating a Charcot-Marie-Tooth type 1A or a Charcot-Marie-Tooth type 1E disease in a patient in need thereof, said method comprising administering to said patient a therapeutically effective amount of an AAV vector according to claim 12, or a pharmaceutical composition comprising said AAV vector and a pharmaceutically acceptable excipient.

22. The method according to claim 21, wherein said AAV vector or said pharmaceutical composition is administered by systemic, intrathecal or intraneural route.


SEQUENCE LISTING (81 sequences)

SEQ ID NO: 1 - RNA, 21 nt, synthetic construct
  source 1..21: mol_type = other RNA
ccctggcccttctcagcggtg

SEQ ID NO: 2 - RNA, 21 nt, synthetic construct
  source 1..21: mol_type = other RNA
cctggcccttctcagcggtgt

SEQ ID NO: 3 - RNA, 21 nt, synthetic construct
  source 1..21: mol_type = other RNA
ctggcccttctcagcggtgtc

SEQ ID NO: 4 - RNA, 21 nt, synthetic construct
  source 1..21: mol_type = other RNA
tggcccttctcagcggtgtca

SEQ ID NO: 5 - RNA, 21 nt, synthetic construct
  source 1..21: mol_type = other RNA
ggcccttctcagcggtgtcat

SEQ ID NO: 6 - RNA, 21 nt, synthetic construct
  source 1..21: mol_type = other RNA
gcccttctcagcggtgtcatc

SEQ ID NO: 7 - RNA, 21 nt, synthetic construct
  source 1..21: mol_type = other RNA
cccttctcagcggtgtcatct

SEQ ID NO: 8 - RNA, 21 nt, synthetic construct
  source 1..21: mol_type = other RNA
ccttctcagcggtgtcatcta

SEQ ID NO: 9 - RNA, 21 nt, synthetic construct
  source 1..21: mol_type = other RNA
cttctcagcggtgtcatctat

SEQ ID NO: 10 - RNA, 21 nt, synthetic construct
  source 1..21: mol_type = other RNA
ttctcagcggtgtcatctatg

SEQ ID NO: 11 - RNA, 21 nt, synthetic construct
  source 1..21: mol_type = other RNA
tctcagcggtgtcatctatgt

SEQ ID NO: 12 - RNA, 21 nt, synthetic construct
  source 1..21: mol_type = other RNA
ctcagcggtgtcatctatgtg

SEQ ID NO: 13 - RNA, 21 nt, synthetic construct
  source 1..21: mol_type = other RNA
tcagcggtgtcatctatgtga

SEQ ID NO: 14 - RNA, 21 nt, synthetic construct
  source 1..21: mol_type = other RNA
cagcggtgtcatctatgtgat

SEQ ID NO: 15 - RNA, 21 nt, synthetic construct
  source 1..21: mol_type = other RNA
agcggtgtcatctatgtgatc

SEQ ID NO: 16 - RNA, 21 nt, synthetic construct
  source 1..21: mol_type = other RNA
gcggtgtcatctatgtgatct

SEQ ID NO: 17 - RNA, 21 nt, synthetic construct
  source 1..21: mol_type = other RNA
cggtgtcatctatgtgatctt

SEQ ID NO: 18 - RNA, 21 nt, synthetic construct
  source 1..21: mol_type = other RNA
ggtgtcatctatgtgatcttg

SEQ ID NO: 19 - RNA, 21 nt, synthetic construct
  source 1..21: mol_type = other RNA
gtgtcatctatgtgatcttgc

SEQ ID NO: 20 - RNA, 21 nt, synthetic construct
  source 1..21: mol_type = other RNA
tgtcatctatgtgatcttgcg

SEQ ID NO: 21 - RNA, 21 nt, synthetic construct
  source 1..21: mol_type = other RNA
gtcatctatgtgatcttgcgg

SEQ ID NO: 22 - RNA, 21 nt, synthetic construct
  source 1..21: mol_type = other RNA
tcatctatgtgatcttgcgga

SEQ ID NO: 23 - RNA, 21 nt, synthetic construct
  source 1..21: mol_type = other RNA
catctatgtgatcttgcggaa

SEQ ID NO: 24 - RNA, 21 nt, synthetic construct
  source 1..21: mol_type = other RNA
atctatgtgatcttgcggaaa

SEQ ID NO: 25 - RNA, 21 nt, synthetic construct
  source 1..21: mol_type = other RNA
tctatgtgatcttgcggaaac

SEQ ID NO: 26 - RNA, 21 nt, synthetic construct
  source 1..21: mol_type = other RNA
ctatgtgatcttgcggaaacg

SEQ ID NO: 27 - RNA, 21 nt, synthetic construct
  source 1..21: mol_type = other RNA
tatgtgatcttgcggaaacgc

SEQ ID NO: 28 - RNA, 21 nt, synthetic construct
  source 1..21: mol_type = other RNA
atgtgatcttgcggaaacgcg

SEQ ID NO: 29 - RNA, 21 nt, synthetic construct
  source 1..21: mol_type = other RNA
tgtgatcttgcggaaacgcga

SEQ ID NO: 30 - RNA, 21 nt, synthetic construct
  source 1..21: mol_type = other RNA
gtgatcttgcggaaacgcgaa

SEQ ID NO: 31 - RNA, 21 nt, synthetic construct
  source 1..21: mol_type = other RNA
tgatcttgcggaaacgcgaat

SEQ ID NO: 32 - RNA, 21 nt, synthetic construct
  source 1..21: mol_type = other RNA
gatcttgcggaaacgcgaatg

SEQ ID NO: 33 - RNA, 21 nt, synthetic construct
  source 1..21: mol_type = other RNA
atcttgcggaaacgcgaatga

SEQ ID NO: 34 - DNA, 21 bp, synthetic construct
  source 1..21: mol_type = other DNA
cctgttcttctgccaactctt

SEQ ID NO: 35 - DNA, 21 bp, synthetic construct
  source 1..21: mol_type = other DNA
ggcaatggacacgcaactgat

SEQ ID NO: 36 - DNA, 21 bp, synthetic construct
  source 1..21: mol_type = other DNA
cggtgtcatctatgtgatctt

SEQ ID NO: 37 - DNA, 21 bp, synthetic construct
  source 1..21: mol_type = other DNA
tgtcgatcatcttcagcattc

SEQ ID NO: 38 - DNA, 21 bp, synthetic construct
  source 1..21: mol_type = other DNA
cacgatcgtcagccaatggat

SEQ ID NO: 39 - RNA, 21 nt, synthetic construct
  source 1..21: mol_type = other RNA
tctcagcggtgtcatctacgt

SEQ ID NO: 40 - RNA, 21 nt, synthetic construct
  source 1..21: mol_type = other RNA
ctcagcggtgtcatctacgtg

SEQ ID NO: 41 - RNA, 21 nt, synthetic construct
  source 1..21: mol_type = other RNA
gcggtgtcatctacgtgatct

SEQ ID NO: 42 - RNA, 21 nt, synthetic construct
  source 1..21: mol_type = other RNA
cggtgtcatctacgtgatctt

SEQ ID NO: 43 - RNA, 21 nt, synthetic construct
  source 1..21: mol_type = other RNA
ggtgtcatctacgtgatcttg

SEQ ID NO: 44 - RNA, 21 nt, synthetic construct
  source 1..21: mol_type = other RNA
tcatctacgtgatcttgcgga

SEQ ID NO: 45 - RNA, 21 nt, synthetic construct
  source 1..21: mol_type = other RNA
caccgctgagaagggccaggg

SEQ ID NO: 46 - RNA, 21 nt, synthetic construct
  source 1..21: mol_type = other RNA
acaccgctgagaagggccagg

SEQ ID NO: 47 - RNA, 21 nt, synthetic construct
  source 1..21: mol_type = other RNA
gacaccgctgagaagggccag

SEQ ID NO: 48 - RNA, 21 nt, synthetic construct
  source 1..21: mol_type = other RNA
tgacaccgctgagaagggcca

SEQ ID NO: 49 - RNA, 21 nt, synthetic construct
  source 1..21: mol_type = other RNA
atgacaccgctgagaagggcc

SEQ ID NO: 50 - RNA, 21 nt, synthetic construct
  source 1..21: mol_type = other RNA
gatgacaccgctgagaagggc

SEQ ID NO: 51 - RNA, 21 nt, synthetic construct
  source 1..21: mol_type = other RNA
agatgacaccgctgagaaggg

SEQ ID NO: 52 - RNA, 21 nt, synthetic construct
  source 1..21: mol_type = other RNA
tagatgacaccgctgagaagg

SEQ ID NO: 53 - RNA, 21 nt, synthetic construct
  source 1..21: mol_type = other RNA
atagatgacaccgctgagaag

SEQ ID NO: 54 - RNA, 21 nt, synthetic construct
  source 1..21: mol_type = other RNA
catagatgacaccgctgagaa

SEQ ID NO: 55 - RNA, 21 nt, synthetic construct
  source 1..21: mol_type = other RNA
acatagatgacaccgctgaga

SEQ ID NO: 56 - RNA, 21 nt, synthetic construct
  source 1..21: mol_type = other RNA
cacatagatgacaccgctgag

SEQ ID NO: 57 - RNA, 21 nt, synthetic construct
  source 1..21: mol_type = other RNA
tcacatagatgacaccgctga

SEQ ID NO: 58 - RNA, 21 nt, synthetic construct
  source 1..21: mol_type = other RNA
atcacatagatgacaccgctg

SEQ ID NO: 59 - RNA, 21 nt, synthetic construct
  source 1..21: mol_type = other RNA
gatcacatagatgacaccgct

SEQ ID NO: 60 - RNA, 21 nt, synthetic construct
  source 1..21: mol_type = other RNA
agatcacatagatgacaccgc

SEQ ID NO: 61 - RNA, 21 nt, synthetic construct
  source 1..21: mol_type = other RNA
aagatcacatagatgacaccg

SEQ ID NO: 62 - RNA, 21 nt, synthetic construct
  source 1..21: mol_type = other RNA
caagatcacatagatgacacc

SEQ ID NO: 63 - RNA, 21 nt, synthetic construct
  source 1..21: mol_type = other RNA
gcaagatcacatagatgacac

SEQ ID NO: 64 - RNA, 21 nt, synthetic construct
  source 1..21: mol_type = other RNA
cgcaagatcacatagatgaca

SEQ ID NO: 65 - RNA, 21 nt, synthetic construct
  source 1..21: mol_type = other RNA
ccgcaagatcacatagatgac

SEQ ID NO: 66 - RNA, 21 nt, synthetic construct
  source 1..21: mol_type = other RNA
tccgcaagatcacatagatga

SEQ ID NO: 67 - RNA, 21 nt, synthetic construct
  source 1..21: mol_type = other RNA
ttccgcaagatcacatagatg

SEQ ID NO: 68 - RNA, 21 nt, synthetic construct
  source 1..21: mol_type = other RNA
tttccgcaagatcacatagat

SEQ ID NO: 69 - RNA, 21 nt, synthetic construct
  source 1..21: mol_type = other RNA
gtttccgcaagatcacataga

SEQ ID NO: 70 - RNA, 21 nt, synthetic construct
  source 1..21: mol_type = other RNA
cgtttccgcaagatcacatag

SEQ ID NO: 71 - RNA, 21 nt, synthetic construct
  source 1..21: mol_type = other RNA
gcgtttccgcaagatcacata

SEQ ID NO: 72 - RNA, 21 nt, synthetic construct
  source 1..21: mol_type = other RNA
cgcgtttccgcaagatcacat

SEQ ID NO: 73 - RNA, 21 nt, synthetic construct
  source 1..21: mol_type = other RNA
tcgcgtttccgcaagatcaca

SEQ ID NO: 74 - RNA, 21 nt, synthetic construct
  source 1..21: mol_type = other RNA
ttcgcgtttccgcaagatcac

SEQ ID NO: 75 - RNA, 21 nt, synthetic construct
  source 1..21: mol_type = other RNA
attcgcgtttccgcaagatca

SEQ ID NO: 76 - RNA, 21 nt, synthetic construct
  source 1..21: mol_type = other RNA
cattcgcgtttccgcaagatc

SEQ ID NO: 77 - RNA, 21 nt, synthetic construct
  source 1..21: mol_type = other RNA
tcattcgcgtttccgcaagat

SEQ ID NO: 78 - DNA, 19 bp, synthetic construct
  source 1..19: mol_type = other DNA
agttccagtacggctccaa

SEQ ID NO: 79 - DNA, 1828 bp, synthetic construct
  source 1..1828: mol_type = other DNA
agttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccgggcagaaactccgctgagcagaacttgccgccagaatgctcctcctgttgctgagtatcatcgtcctccacgtcgcggtgctggtgctgctgttcgtctccacgatcgtcagccaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactca

SEQ ID NO: 80 - DNA, 53 bp, synthetic construct
  source 1..53: mol_type = other DNA
ccctggcccttctcagcggtgtcatctatgtgatcttgcggaaacgcgaatga

SEQ ID NO: 81 - DNA, 12 bp, synthetic construct
  source 1..12: mol_type = other DNA
atcgatacgtag